(19) United States
(12) Patent Application Publication
(10) Pub. No.: US 2021/0038503 A1
(43) Pub. Date: Feb. 11, 2021

(54) COMPOSITIONS AND METHODS FOR TOPICAL TREATMENT OF DERMAL AND OCULAR CONDITIONS

(71) Applicant: GABADERM, INC., La Jolla, CA (US)

(72) Inventor: John DOBAK, III, La Jolla, CA (US)

(21) Appl. No.: 16/966,799

(22) PCT Filed: Jan. 31, 2019
(86) PCT No.: PCT/US2019/015916

§ 371 Date: Jul. 31, 2020

Related U.S. Application Data
(60) Provisional application No. 62/624,481, filed on Jan. 31, 2018.
(60) Provisional application No. 62/640,506, filed on Mar. 8, 2018.
(60) Provisional application No. 62/716,203, filed on Aug. 8, 2018.

(51) Int. Cl. A61K 9/00; A61K 31/05; A61K 9/107; A61K 9/08; A61K 9/06; A61K 9/127; A61P 17/06; A61P 17/04

(52) U.S. Cl. CPC A61K 9/0014; A61K 31/05; A61K 9/107; A61K 9/08; A61K 45/06; A61K 9/06; A61K 9/127; A61P 17/06; A61P 17/04; A61K 9/0048

(57) ABSTRACT

The instant disclosure provides methods and compositions for the treatment of skin disorders associated with itch, irritation, inflammation, and pain, such as psoriasis, eczema, and skin ulcers. The instant disclosure also provides methods and compositions for the treatment of ocular conditions associated with itch, irritation, inflammation, and pain, such as ocular itch, conjunctivitis and eye infections. Generally, compositions include modulator of gamma-amino-butyric-acid (GABA) receptor, such as propofol. The concentration of the GABA modulator in the composition is typically sufficiently high so as to activate GABA receptors in nerve and immune cells of the skin, but sufficiently low so as to not result in any significant systemic exposure.

### CROSS-REFERENCE

This application claims the benefit of U.S. Provisional Application No. 62/624,481, filed Jan. 31, 2018, U.S. Provisional Application No. 62/640,506, filed Mar. 8, 2018, and U.S. Provisional Application No. 62/716,203, filed Aug. 8, 2018, each of which is incorporated herein by reference in its entirety.

### BACKGROUND OF THE DISCLOSURE

GABA_{A}R and GABA_{B}R receptors are activated by gamma-amino-butyric-acid (GABA). GABA_{A}R is a chloride ion channel receptor. In neurons, GABA_{A}R activity hyperpolarizes or depolarizes nerve cells resulting in reduced or temporary increased neuronal activity. GABA_{B}R is a G-coupled protein receptor which modulates intracellular potassium and calcium via cyclic AMP. GABA_{B}R can have inhibitory effects in neurons by down regulating the effects of other receptors (e.g., TRPV receptor or capsaicin receptor responses at synapses or terminal nerve endings). GABA receptors A and B are also found on immune cells: T-cells, monocytes, and macrophages. Activation of GABA receptors in these cells attenuates their activity, including: reducing proliferation, inhibiting cytokine production, and limiting cell migration.

The GABA_{A}R is made up of 5 subunits 2 alpha, 2 beta, and 1 gamma. In some central nervous areas the beta subunit is replaced with the delta. GABA_{A}R ligands bind to different subunits to activate GABA_{A}R or potentiate GABA_{A}R activation. GABA_{A}R's natural ligand is GABA. GABA binds the alpha and beta sub units. Direct agonists such as muscimol bind to the GABA site on the receptor and cause channel opening. GABA_{A}R also has potentiator (allosteric modulators) binding sites. Allosteric modulators such as benzodiazepines bind to this site and increase the receptor activation when GABA or a direct agonist binds. Many pharmaceutical products that affect the GABA system are allosteric modulators, e.g., barbiturates, zolpidem, and valium.

### SUMMARY OF THE DISCLOSURE

There are numerous skin and ocular conditions that are accompanied by pain, itching, irritation, and inflammation at affected areas. Patients suffering from these disorders would benefit from localized, topical treatments that alleviate these symptoms. Many skin diseases are inflammatory with significant T-cell activation, proliferation, and cytokine production. Itch and other neural mediated effects are significant components and contributors to skin diseases as well.

GABA receptors (GABA-R), both GABA_{A}R and GABA_{B}R, are found in free terminal nerve endings of A-delta and C fibers in the skin. A-delta and C fibers are involved in itch and pain sensation. Activation of GABA_{A}R and GABA_{B}R in these fibers can down regulate their activity, thereby suppressing itch and pain. GABA_{A}R are also found on immune cells located in the skin, e.g., T cells. GABA_{A}R activation may modulate the activity of these immune cells. For instance, GABA_{A}R activation may reduce proliferation of T cells, reduce activation of T cells, reduce cytokine release and production, modulate activation of antigen presenting cells, and modulate macrophage migration. Thus, GABA_{A}R modulation can reduce both the inflammatory and neural mediated aspects of the disease. In contrast, GABA_{B}R is not found on immune cells of the skin and do not modulate immune cell activity in the skin.

Patients with skin disorders may benefit from treatment with GABA-R modulators due to their aforementioned effects on nerves and immune cells in the skin. However, systemic exposure to GABA-R agonists may cause sedation, which would be an undesirable effect for most of these patients. Furthermore, GABA-R agonists can be addictive. A non-limiting example of a GABA-R agonist is propofol. Propofol is widely used as an intravenous anesthetic with excellent safety. Propofol has demonstrated anesthetic, analgesic, antinociceptive, and anti-inflammatory properties in mammals. Unlike many other GABA-R agonists, propofol can interact with a beta subunit of GABA_{A}R on immune cells and terminal nerve endings of the skin. However, propofol is largely viewed as a short acting anesthetic and major sedative. Proprofol is also known to cause pain and itching when administered intravenously for anesthetic purposes and is the main drawback of the drug. This effect is thought to be mediated by Kallikrien/Kinin activation and TRPV receptor activation in nerve endings in tissue. These TRPV receptors are thought to be a main cause of itch and sensitivities of the skin. For at least the foregoing reasons, propofol would not necessarily be a logical choice for topical GABA modulation. Accordingly, those skilled in the art would not typically consider it as a drug suitable for treatment of irritated skin because of the risk of systemic exposure and undesirable sedative effects.

Use of GABA receptors agonists to treat inflamed skin, such as skin having an allergic reaction or dermatitis (psoriasis, atopic, contact, etc.), requires modulation of immune cells, particularly T-cells, as well as terminal nerve endings. Consequently inflamed skin benefits from a GABA agonist, such as propofol, that binds a beta subunit of GABA_{A}R on immune cells, as well as terminal nerve endings of the skin. Modulation of T-cells in the skin requires a potent full agonist since endogenous agonist GABA may not be present in concentrations sufficient for allosteric modulation by other drugs. To effectively accomplish this, a GABA_{A}R agonist that binds and fully activates a beta subunit is ideal, particularly the beta-2 and 3 subunits. Ideally the GABA_{A}R agonist also functions as a GABA_{B}R agonist in order to modulate activity of macrophages in the skin that express only GABA_{B}R, (no GABA_{A}R). Modulation of macrophage GABA_{B}R in the skin may down regulate macrophage migration and/or production of macrophage inflammatory mediators. Propofol, an agonist of both GABA_{A}R beta subunits and GABA_{B}R, uniquely meets all of these requirements.

Methods and compositions are disclosed herein for treating skin disorders by topically delivering GABA-R modulators, predominantly to the skin or ocular region while avoiding the aforementioned, undesirable effects. In many instances, the GABA-R modulator is formulated in a composition to act as a full agonist of GABA_{A}R as well as an agonist of GABA_{B}R, thereby having desirable effects in immune cells (e.g., T cells and macrophages), and terminal nerve endings. Topical applications of compositions disclosed herein that comprise GABA-R modulators, such as propofol, can provide sustained activity in the epidermis without major sedative and anesthetic affects. In some instances, there are minimal sedative or anesthetic effects. For example a subject receiving a topical application as disclosed herein may be slightly relaxed, but can still carry on their normal daily activities. In some instances, there are no sedative or anesthetic effects. Compositions and methods disclosed herein do not result in pain and itch, but rather ameliorates these sensations.

GABA-R modulator, propofol, can act as both an allosteric modulator and a direct agonist of GABA_{A}R in nerve cells and immune cells of the skin. Propofol binds the beta subunit at the transmembrane region of GABA_{A}R. Propofol acts as a potentiator of GABA at low concentrations and a direct activator of GABA_{A}R at high concentrations. Propofol also activates GABA_{B}R, especially at higher concentrations. GABA-R agonists are not normally present in the skin (in contrast to the CNS). Therefore, methods and compositions disclosed herein provide propofol at concentrations sufficiently high to have direct GABA-R agonist activity locally in the skin with minimal effects elsewhere. In some instances, methods comprise applying a dose of propofol to the skin that is sufficiently high for propofol to act as a direct agonist of GABA_{A}R. Propofol also has the unique effect of inhibiting the breakdown of endocannabinoids which increases their level locally. Propofol inhibits fatty acid amide hydrolase, which may increase the levels of anandamide and 2-arachidonoylglycerol endocannabinoids, thereby reducing inflammation and itch. Propofol may also attenuate neuropathic pain due to effects on cutaneous nerves in the skin. Examples include diabetic neuropathy and post-herpetic neuralgia.

Propofol is particularly advantageous for pharmaceutical compositions and methods disclosed herein because it has a long half-life and there are limited metabolizers of propofol in the skin. Thus, propofol provides a suitable duration of action for a dermatologic or ocular medical product. Patients are also less likely to develop tolerance or tachyphylaxis to propofol relative to other GABA agonists. Propofol modulates other systems, such as the endocannabinoid system, which plays a role in skin diseases and contributes to their severity. Thus, propofol may be effective for the treatment of skin disorders by modulating activity of receptors other than GABA-Rs. Furthermore, propofol is a lipophilic small molecule ideal for absorption into the epidermis and for topical formulations. Generally, molecules larger than 500 Daltons will not penetrate the skin.

Disclosed herein, in some aspects, are methods for treating a condition of the skin on a subject in need thereof comprising applying a therapeutic agent in a suitable carrier to an affected area of the skin, wherein the therapeutic agent is present in the suitable carrier at a concentration sufficient to act as an agonist of a gamma-amino-butyric-acid type A receptor (GABA_{A}R) beta subunit. In some instances, the therapeutic agent acts as an agonist of GABA_{A}R beta subunit in the absence of gamma-amino-butyric-acid (GABA). In some instances, the therapeutic agent acts as an agonist of a gamma-amino-butyric-acid type B receptor (GABA_{B}R). In some instances, the therapeutic agent is lipophilic. In some instances, the therapeutic agent is present in the suitable carrier at a concentration sufficient to behave as inhibitor of a fatty acid amide hydrolase. In some instances, the therapeutic agent does not bind an alpha subunit of GABA_{A}R. In some instances, the therapeutic agent does not bind a gamma subunit of GABA_{B}R. In some instances, the therapeutic agent is non-steroidal. In some instances, the therapeutic agent has a molecular weight of 500 Daltons or less. In some instances, the therapeutic agent comprises propofol. In some instances, the skin condition comprises inflammation. In some instances, the skin condition is selected from dermatitis, psoriasis, eczema, hidradenitis suppurativa, and rosacea. In some instances, dermatitis is selected from atopic dermatitis, contact dermatitis and seborrheic dermatitis. In some instances, inflammation is associated with a Th2 mediated inflammatory disease. In some instances, the condition of the skin comprises inflammation. In some instances, the condition of the skin comprises skin itch. In some instances, the condition of the skin comprises pain of the skin. In some instances, the pain is associated with diabetic neuropathy or post herpetic neuralgia. In some instances, the condition of the skin comprises a skin ulcer. In some instances, the skin ulcer is a pressure ulcer. In some instances, the skin ulcer is a vascular ulcer. In some instances, the condition of the skin comprises a skin wound. In some instances, the therapeutic agent does not activate or potentiate a GABA_{A} receptor on an immune cell of the subject other than an immune cell in the skin. In some instances, the therapeutic agent does not penetrate the skin of the subject beyond the epidermis. In some instances, the therapeutic agent reduces proliferation of T cells in the skin. In some instances, the therapeutic agent reduces activation of T cells in the skin. In some instances, the therapeutic agent reduces at least one of cytokine release and cytokine production by T cells of in the skin. In some instances, the therapeutic agent modulates activation of antigen presenting cells in the skin. In some instances, the therapeutic agent modulates macrophage migration in the skin. In some instances, the concentration of the therapeutic agent in the pharmaceutical composition is about 1% w/w to about 25% w/w. In some instances, the concentration of the therapeutic agent in the suitable carrier is about 5% w/w to about 20% w/w. In some instances, methods comprise applying the therapeutic agent more than once a day. In some instances, methods comprise applying the therapeutic agent more than twice a day.

Further disclosed herein, in some aspects are methods for treating an ocular condition of a subject in need thereof comprising applying a therapeutic agent in a suitable carrier to an affected area of an eye of the subject, wherein the therapeutic agent is present in the suitable carrier at a concentration sufficient to act as an agonist of a GABA_{A}R beta subunit located on a cell in the affected eye area. In some instances, the therapeutic agent acts as an agonist of GABA_{A}R beta subunit in the absence of gamma-amino-butyric-acid (GABA). In some instances, the therapeutic agent acts as an agonist of a gamma-amino-butyric-acid type B receptor (GABA_{B}R). In some instances, the agonist is lipophilic. In some instances, the therapeutic agent is present in the suitable carrier at a concentration sufficient to behave as inhibitor of a fatty acid amide hydrolase. In some instances, the therapeutic agent does not bind an alpha subunit of GABA_{A}R. In some instances, the therapeutic agent does not bind an gamma subunit of GABA_{B}R. In some instances, the therapeutic agent is non-steroidal. In some instances, the therapeutic agent has a molecular weight of 500 Daltons or less. In some instances, the therapeutic agent is propofol. In some instances, the suitable carrier comprises a colloid. In some instances, the composition is in the form of an aqueous-oil emulsion. In some instances, the ocular condition is selected from ocular itch, ocular inflammation, a scratched cornea, conjunctivitis, an eye infection, and a combination thereof. In some instances, applying comprises administering drops of the composition to the eye. In some instances, the suitable carrier is an ointment and applying comprises dabbing the composition along an edge or corner of an eyelid. In some instances, a plasma concentration of the therapeutic agent in the subject does not exceed 5 ng/ml.

Disclosed herein, in some aspects, are compositions comprising a therapeutic agent in a suitable carrier for topical application, wherein the therapeutic agent is present in the suitable carrier at a concentration sufficient to act as an agonist of a GABA_{A}R beta subunit. In some instances, the therapeutic agent acts as an agonist of GABA_{A}R beta subunit in the absence of gamma-amino-butyric-acid (GABA). In some instances, the therapeutic agent acts as an agonist of a gamma-amino-butyric-acid type B receptor (GABA_{B}R). In some instances, the therapeutic agent is lipophilic. In some instances, the therapeutic agent is present in the suitable carrier at a concentration sufficient to behave as inhibitor of a fatty acid amide hydrolase. In some instances, the therapeutic agent does not bind an alpha subunit of GABA_{A}R. In some instances, the therapeutic agent does not bind a gamma subunit of GABA_{B}R. In some instances, the therapeutic agent is non-steroidal. In some instances, the therapeutic agent has a molecular weight of 500 Daltons or less. In some instances, the therapeutic agent comprises propofol. In some instances, the therapeutic agent does not activate or potentiate a GABA_{A} receptor on an immune cell of the subject other than an immune cell in the skin. In some instances, the therapeutic agent does not penetrate the skin of the subject beyond the epidermis. In some instances, the therapeutic agent reduces proliferation of T cells in the skin. In some instances, the therapeutic agent reduces activation of T cells in the skin. In some instances, the therapeutic agent reduces at least one of cytokine release and cytokine production by T cells of in the skin. In some instances, the therapeutic agent modulates activation of antigen presenting cells in the skin. In some instances, the therapeutic agent modulates macrophage migration in the skin. In some instances, the concentration of the therapeutic agent in the pharmaceutical composition is about 1% w/w to about 25% w/w. In some instances, the concentration of the therapeutic agent in the suitable carrier is about 5% w/w to about 20% w/w. In some instances, the suitable carrier is selected from at least one of a cream, an emulsion, an ointment, a microparticle, a nanoparticle, a suspension, a gel, a lotion, and a patch. In some instances, the composition comprises an additional active ingredient selected from an anti-inflammatory agent, an analgesic agent, an anti-itch agent, and a combination thereof. In some instances, the composition does not comprise a skin penetration enhancer. In some instances, the composition does not comprise at least one of ethanol, methanol, DMSO, terpene, and SDS. In some instances, the composition contains mineral oil, a triglyceride, lanolin, and a steroid. In some instances, the composition contains mineral oil. In some instances, the suitable carrier comprises at least one of a lipid nanoparticles and a liposome. In some instances, the suitable carrier maintains the composition as a solid at body temperature. In some instances, the composition is formulated for administration to the eye. In some instances, the suitable carrier comprises a colloid. In some instances, the composition is in the form of an aqueous-oil emulsion. In some instances, the therapeutic agent has a log partitioning coefficient greater than 4. In some instances, the composition comprises a GABA_{B}R modulator. In some instances, the composition comprises a TRP channel modulator.

Further disclosed herein, in some aspects, are methods for treating an inflammatory condition of the skin of a subject, the method comprising applying propofol in a suitable carrier to an affected area of the skin. In some instances, the inflammatory condition comprises cytokine mediated inflammation, Th2 mediated inflammation, or a combination thereof. In some instances, the inflammatory condition is selected from dermatitis psoriasis, eczema, hidradenitis suppurativa, rosacea, herpetic neuralgia, and a combination thereof. In some instances, dermatitis comprises atopic dermatitis. In some instances, dermatitis comprises contact dermatitis. In some instances, dermatitis comprises seborrheic dermatitis. In some instances, the suitable carrier is selected from a cream and an ointment. In some instances, the suitable carrier comprises less than 10% w/w of a penetration enhancer. In some instances, the concentration of propofol in the carrier is 0.1%-20% w/w. In some instances, methods comprise applying propofol in the suitable carrier 1 to 3 times per day.

Disclosed herein, in some aspects, are methods for treating an inflammatory condition of the skin of a subject, the method comprising applying propofol and a GABA_{B}R modulator to an affected area of the skin. In some instances, the GABA_{B}R modulator enhances, increases or promotes activity of GABA_{B}R. In some instances, the GABA_{B}R modulator is a GABA_{B}R agonist. In some instances, the propofol and GABA_{B}R modulator are applied as a single composition. In some instances, the concentration of propofol in the single composition is 0.1%-20% w/w. In some instances, the concentration of GABA_{B}R modulator in the single composition is 0.1%-10% w/w. In some instances, the propofol is delivered in a first composition and the GABA_{B}R modulator is applied in a second composition. In some instances, the first composition has a different pharmaceutical carrier than the second composition. In some instances, at least one of the first composition and the second composition contains less than 10% w/w of a penetration enhancer. In some instances, the concentration of propofol in the first composition is 0.1%-20% w/w. In some instances, the concentration of GABA_{B}R modulator in the second composition is 0.1%-10% w/w. In some instances, the inflammatory condition comprises Th2 mediated inflammation. In some instances, the inflammatory condition is selected from dermatitis psoriasis, eczema, hidradenitis suppurativa, rosacea, herpetic neuralgia, and a combination thereof. In some instances, dermatitis comprises atopic dermatitis. In some instances, dermatitis comprises contact dermatitis. In some instances, dermatitis comprises seborrheic dermatitis. In some instances, at least one of the propofol and the GABA_{B}R modulator is formulated as a cream or an ointment. In some instances, the GABA_{B}R modulator is baclofen. In some instances, the GABA_{B}R modulator is phenibut. In some instances, applying does not occur more than 3 times per day.

Further disclosed herein, in some aspects, are methods for treating an inflammatory condition of the skin of a subject, the methods comprising applying a GABA_{A}R modulator and a GABA_{B}R modulator to an affected area of the skin. In some instances, the GABA_{B}R modulator enhances, increases or promotes activity of GABA_{B}R. In some instances, the GABA_{B}R modulator is a GABA_{B}R agonist. In some instances, the GABA_{A}R modulator and GABA_{B}R modulator are applied as a single composition. In some instances, the concentration of GABA_{A}R modulator in the single composition is 0.1%-20% w/w. In some instances, the concentration of GABA_{B}R modulator in the single composition is 0.1%-10% w/w. In some instances, the GABA_{A}R modulator is delivered in a first composition and the GABA_{B}R modulator is applied in a second composition. In some instances, the first composition has a different pharmaceutical carrier than the second composition. In some instances, at least one of the first composition and the second composition contains less than 10% w/w of a penetration enhancer. In some instances, the concentration of GABA_{A}R modulator in first composition is 0.1%-20% w/w. In some instances, the concentration of GABA_{B}R modulator in second composition is 0.1%40% w/w. In some instances, the inflammatory condition comprises Th2 mediated inflammation. In some instances, the inflammatory condition is selected from dermatitis psoriasis, eczema, hidradenitis suppurativa, rosacea, herpetic neuralgia, and a combination thereof. In some instances, dermatitis comprises atopic dermatitis. In some instances, dermatitis comprises contact dermatitis. In some instances, dermatitis comprises seborrheic dermatitis. In some instances, at least one of the GABA_{A}R and the GABA_{B}R modulator is formulated as a cream or an ointment. In some instances, the GABA_{B}R modulator is baclofen. In some instances, the GABA_{B}R modulator is phenibut. In some instances, applying does not occur more than 3 times per day. In some instances, the GABA_{A}R modulator is a GABA_{A}R agonist. In some instances, the GABA_{A}R modulator is a propofol. In some instances, the GABA_{A}R modulator is a barbiturate. In some instances, the GABA_{A}R modulator is a zolpidem.

Disclosed herein, in some aspects, are methods for treating an inflammatory condition of the skin of a subject, the methods comprising applying a GABA_{A}R modulator and a TRP channel modulator to an affected area of the skin. In some instances, the TRP channel modulator and GABA_{A}R modulator are applied as a single composition. In some instances, the concentration of GABA_{A}R modulator in the single composition is 0.1%-20% w/w. In some instances, the concentration of TRP channel modulator in the single composition is 0.1%-10% w/w. In some instances, the GABA_{A}R modulator is delivered in a first composition and the TRP channel modulator is applied in a second composition. In some instances, the first composition has a different pharmaceutical carrier than the second composition. In some instances, at least one of the first composition and the second composition contains less than 10% w/w of a penetration enhancer. In some instances, the concentration of GABA_{A}R modulator in the first composition is 0.1%-20% w/w. In some instances, the concentration of TRP channel modulator in the second composition is 0.1%-10% w/w. In some instances, the inflammatory condition comprises Th2 mediated inflammation. In some instances, the inflammatory condition is selected from dermatitis psoriasis, eczema, hidradenitis suppurativa, rosacea, herpetic neuralgia, and a combination thereof. In some instances, dermatitis comprises atopic dermatitis. In some instances, dermatitis comprises contact dermatitis. In some instances, dermatitis comprises seborrheic dermatitis. In some instances, at least one of the GABA_{A}R modulator and the TRP channel modulator is formulated as a cream or an ointment. In some instances, the TRP channel modulator is camphor. In some instances, the TRP channel modulator is menthol. In some instances, applying occurs more than 2 times per day. In some instances, the GABA_{A}R modulator is a GABA_{A}R agonist. In some instances, the GABA_{A}R modulator is a propofol. In some instances, the GABA_{A}R modulator is a barbiturate. In some instances, the GABA_{A}R modulator is a zolpidem. In some instances, the TRP channel modulator is a TRP channel antagonist. In some instances, the TRP channel modulator modulates the activity of a TRP channel selected from TRPA1, TRPV1 and TRPM8. In some instances, methods comprise applying a GABA_{B}R modulator to the affected area of the skin. In some instances, the GABA_{B}R modulator, GABA_{A}R modulator, and TRP channel modulator are applied as a single pharmaceutical composition. In some instances, the GABA_{B}R modulator is applied before at least one of the GABA_{A}R modulator and the TRP channel modulator. In some instances, the GABA_{B}R modulator is applied after at least one of the GABA_{A}R modulator and the TRP channel modulator.

Further disclosed herein, in some aspects, are methods for treating skin itch of a subject comprising applying a GABA_{A}R modulator and a GABA_{B}R modulator to affected areas of the skin. In some instances, the GABA_{A}R modulator is propofol. In some instances, the GABA_{B}R modulator and GABA_{A}R modulator are applied as a single composition. In some instances, the concentration of GABA_{A}R modulator in the single composition is 0.1%-20% w/w. In some instances, the concentration of GABA_{B}R modulator in the single composition is 0.1%40% w/w. In some instances, the GABA_{A}R modulator is delivered in a first composition and the GABA_{B}R modulator is applied in a second composition. In some instances, the concentration of GABA_{A}R modulator in the first composition is 0.1%-20% w/w. In some instances, the concentration of GABA_{B}R modulator in the second composition is 0.1%-10% w/w. In some instances, the first composition has a different pharmaceutical carrier than the second composition. In some instances, at least one of the GABA_{A}R modulator and GABA_{B}R modulator is applied in the form selected from a cream, emulsion, ointment, nanoparticle, lotion, patch, and a combination thereof.

### Certain Terminologies

Unless defined otherwise, all technical and scientific terms used herein have the same meaning as is commonly understood by one of skill in the art to which the claimed subject matter belongs. It is to be understood that the foregoing general description and the following examples are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. For example, “about 5 μL” means “about 5 μL” and also “5 μL.” Generally, the term “about” includes an amount that would be expected to be within experimental or clinical application error. The term “about” includes values that are within 10% less to 10% greater of the value provided. For example, “about 50%” means “between 45% and 55%.” Also, by way of example, “about 30” means “between 27 and 33.”

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Alternatively, treatment is “effective” if the progression of a disease is reduced or halted. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management.

Propofol includes analogs of propofol with substantially the same receptor activity and pro-drugs on propofol, such as fospropofol, that can be metabolized to release propofol. By way of example, fospropofol is metabolized by alkaline phosphatase to release propofol. Alkaline phosphatase may be found in the tissues of the skin or eye.

The section headings used herein are for organizational purposes only and are not to be construed as limiting the subject matter described.

### BRIEF DESCRIPTION OF THE DRAWINGS

FIG. 1 shows the chemical structure of propofol, an agonist of a gamma-amino-butyric-acid type A receptor (GABA_{A}R).

FIG. 2 shows the chemical structure of phenibut, an agonist of a gamma-amino-butyric-acid type B receptor (GABA_{B}R).

FIG. 3 shows the chemical structure of baclofen, an agonist of a gamma-amino-butyric-acid type B receptor (GABA_{B}R).

FIG. 4 shows the chemical structure of camphor, an antagonist of a transient receptor potential A1 (TRPA1) channel.

FIG. 5 shows the chemical structure of menthol, a TRPA1 and TRPV1 channel modulator. Menthol may also be act as a TRPM8 Receptor agonist.

FIG. 6 shows topical propofol treatment reduces skin inflammation in a mouse model.

FIG. 7 shows psoriatic lesions were reduced with topical application of propofol in a cream based formulation.

FIG. 8 shows a gutate psoriatic lesion reduced and resolved by topical application of 10% propofol in cream.

FIG. 9 shows a gutate psoriatic lesion resolved by topical application of 10% propofol in cream.

FIG. 10 shows a plaque psoriatic lesion resolved by topical application of 10% propofol in cream.

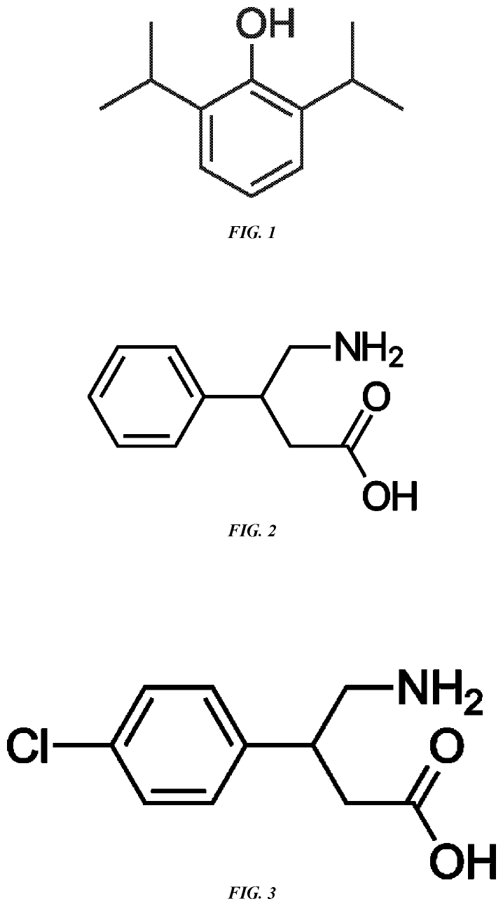

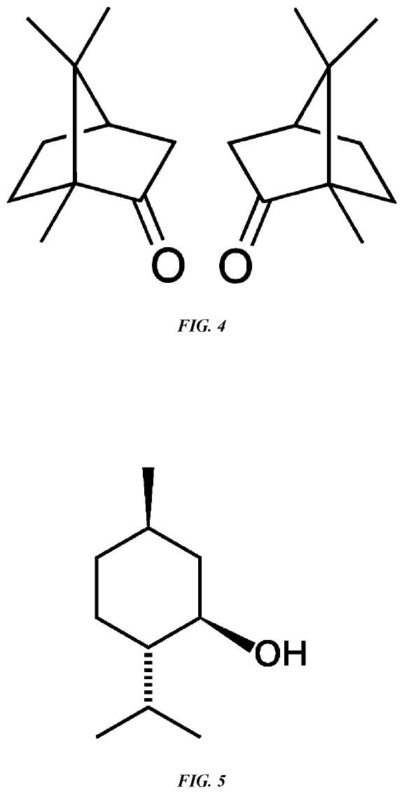

### DETAILED DESCRIPTION OF THE DISCLOSURE

Therapeutic Platforms

Disclosed herein, in some aspects, are methods for treating conditions of the skin in a subject in need thereof that comprise applying a therapeutic agent in a suitable carrier to an affected area of the skin. The therapeutic agent is typically a GABA-R modulator. Also disclosed herein, in some aspects are methods of treating conditions of the eye comprise applying a therapeutic agent in a suitable carrier to an eye. In general, therapeutic agents disclosed herein are capable of modulating a GABA receptor's activity. By way of non-limiting example, a modulator of a GABA-R may comprise a GABA-R agonist. In some instances, the GABA-R agonist acts as an agonist of GABA_{A} receptor. In some instances, the GABA-R agonist is propofol. In some instances, the GABA-R agonist is a synthetic derivative of propofol. In some instances, the GABA-R agonist is a synthetic derivative of propofol, wherein the synthetic derivative is propofol substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino. Non-limiting examples of suitable carriers are creams, emulsions, ointments, microparticles, nanoparticles, suspensions, gels, and lotions.

Additional components and features of compositions suitable for such methods are further described herein.

In some instances, methods disclosed herein comprise treating a condition with a single GABA-R modulator. In some instances, methods disclosed herein comprise treating a condition with a not more than one GABA-R modulator. In some instances, methods disclosed herein comprise treating a condition with a not more than two GABA-R modulators. In some instances, methods disclosed herein comprise treating a condition with a single therapeutic agent, wherein the single therapeutic agent is a GABA-R modulator. In some instances, methods disclosed herein comprise treating a condition with a not more than one therapeutic agent. In some instances, methods disclosed herein comprise treating a condition with a not more than two therapeutic agents. In some instances, methods comprise administering a GABA-R modulator and no other therapeutic agents or active ingredients. In some instances, the GABA-R modulator is a GABA-R agonist. In some instances, the GABA-R modulator acts as a GABA_{A}-R agonist. In some instances, the GABA-R modulator acts as a GABA_{A}-R allosteric modulator. In some instances, the GABA-R modulator is propofol or a synthetic derivative thereof.

In some instances, the GABA-R modulator is a propofol derivative. In some instances, the GABA-R modulator is a synthetic propofol derivative selected from fospropofol, HX0969w, PF0713, AZD-3043, and phaxan (alphaxalone).

In some instances, the GABA-R modulator is fospropofol. In some instances, the GABA-R agonist is a synthetic derivative of fospropofol. In some instances, the GABA-R agonist is a synthetic derivative of fospropofol, wherein the synthetic derivative is fospropofol substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, the GABA-R modulator is HX0969w. In some instances, the GABA-R agonist is a synthetic derivative of HX0969w. In some instances, the GABA-R agonist is a synthetic derivative of HX0969w, wherein the synthetic derivative is HX0969w substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, the GABA-R modulator is PF0713. In some instances, the GABA-R agonist is a synthetic derivative of PF0713. In some instances, the GABA-R agonist is a synthetic derivative of PF0713, wherein the synthetic derivative is PF0713 substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, the GABA-R modulator is AZD-3043. In some instances, the GABA-R agonist is a synthetic derivative of AZD-3043. In some instances, the GABA-R agonist is a synthetic derivative of AZD-3043, wherein the synthetic derivative is AZD-3043 substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, the GABA-R modulator is phaxan (alphaxalone). In some instances, the GABA-R agonist is a synthetic derivative of phaxan (alphaxalone). In some instances, the GABA-R agonist is a synthetic derivative of phaxan (alphaxalone), wherein the synthetic derivative is phaxan (alphaxalone) substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, the GABA-R modulator is HX0507. In some instances, the GABA-R agonist is a synthetic derivative of HX0507. In some instances, the GABA-R agonist is a synthetic derivative of HX0507, wherein the synthetic derivative is HX0507 substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, methods disclosed herein comprise treating inflammation of the skin with a pharmaceutical composition disclosed herein. In some instances, methods disclosed herein comprise treating irritation of the skin with a pharmaceutical composition disclosed herein. Irritation of the skin may comprise swelling, redness, pain, or a combination thereof. In some instances, methods disclosed herein comprise treating itch of the skin with a pharmaceutical composition disclosed herein. There are many causes of skin irritation, inflammation and itch. Any one of inflammation, irritation and itching may be due to a skin condition disclosed herein. Any one of inflammation, irritation and itching may be due to an allergy, a drug side effect, an infection, and a food-borne pathogen. Any one of inflammation, irritation and itching may be due to an injury. Non-limiting examples of injuries are bites and burns. These examples are not meant to be limiting. They are provided in order to aid the reader in understanding the utility of the methods and compositions disclosed herein.

In some instances, methods disclosed herein comprise treating inflammation of the skin with a pharmaceutical composition disclosed herein, wherein the inflammation is associated with a cytokine mediated inflammatory condition. In some instances, the cytokine mediated inflammatory condition is mediate by Th2 cells (otherwise referred to as a Th2 cell mediated inflammatory disease). In some instances, the cytokine mediated inflammatory condition comprises dermatitis. In some instances, the cytokine mediated inflammatory condition comprises psoriasis. In some instances, the cytokine mediated inflammatory condition comprises eczema. In some instances, the cytokine mediated inflammatory condition comprises hidradenitis suppurativa. In some instances, the cytokine mediated inflammatory condition comprises rosacea. In some instances, dermatitis comprises atopic dermatitis. In some instances, dermatitis comprises contact dermatitis. In some instances, dermatitis comprises seborrheic dermatitis.

In some instances, methods disclosed herein comprise treating skin itch on a subject in need thereof with a pharmaceutical composition disclosed herein. In some instances, methods disclosed herein comprise treating skin irritation on a subject in need thereof. In some instances, methods disclosed herein comprise treating skin pain on a subject in need thereof. In some instances, the methods comprise applying a GABA-R modulator to an affected of the subject. In some instances, the methods comprise applying propofol to an affected of the subject.

In some instances, methods disclosed herein comprise treating a skin wound on a subject in need thereof with a pharmaceutical composition disclosed herein. In some instances, methods disclosed herein comprise treating a skin ulcer on a subject in need thereof. In some instances, the ulcer is a pressure ulcer. In some instances, the ulcer is a vascular ulcer.

In some instances, methods and compositions disclosed herein are used to treat neuropathic pain. In some instances, methods and compositions disclosed herein are used to treat diabetic neuropathy. In some instances, methods and compositions disclosed herein are used to treat post herpetic neuralgia.

In some instances, methods disclosed herein comprise treating an ocular condition in a subject in need thereof with a pharmaceutical composition disclosed herein. Methods may comprise applying a composition comprising a therapeutic agent disclosed herein in a suitable carrier to an affected area of an eye of the subject. In some instances, the therapeutic agent is a GABA-R modulator. In some instances, the GABA-R modulator is a GABA-R agonist. In some instances, the GABA-R modulator is a GABA_{A} receptor agonist. In some instances, the GABA-R modulator is propofol or a synthetic derivative thereof. By way of non-limiting example, the ocular condition may be selected from ocular itch, ocular inflammation, a scratched cornea, conjunctivitis, an eye infection, and a combination thereof.

In some instances, methods of treating an ocular condition comprise administering drops of a composition to the eye, wherein the composition comprises a therapeutic agent disclosed herein and at least one suitable carrier. Generally, the composition is isotonic with human intraocular fluid. In some instances, methods of treating an ocular condition comprise administering a composition around the eye. For example, methods may comprise applying the composition to an edge of an eyelid or a corner of an eye. In instances when the methods comprise administering a composition around an eye, the composition, by way of non-limiting example, may be provided in the form of a wax, ointment, paste or foam. Formulations with suitable carriers for ocular administration are further described herein.

In some embodiments, the methods may comprise administering a pharmaceutical composition disclosed herein immediately before sleep or before a period of time in which the subject may maintain eye closure. In some embodiments, methods comprise instructing the subject to keep their eyes closed to maintain eye closure for at least 1 minute, at least 5 minutes, at least 10 minutes, at least 15 minutes, at least 20 minutes, at least 30 minutes, at least 1 hour, at least 2 hours, at least 4 hours, or at least 8 hours after the pharmaceutical composition is administered. Methods may comprise instructing the subject to keep their eyes closed from 1 minute to 8 hours after the pharmaceutical composition is administered. Methods may comprise instructing the subject to keep their eyes closed from 1 minute to 2 hours after the pharmaceutical composition is administered. Methods may comprise instructing the subject to keep their eyes closed from 1 minute to 30 minutes after the pharmaceutical composition is administered. Methods may comprise administering an eye cover (e.g., bandage, tape, patch) to the subject after administering a pharmaceutical composition disclosed herein to the eye of the subject.

Methods disclosed herein generally comprise administering only enough of a therapeutic agent to be effective in treating a skin or eye disorder, neuropathic pain, or chronic wound, without having any systemic effects on the subject. In some instances, methods do not result in transdermal delivery of a therapeutic agent disclosed herein. For instance, methods may comprise topically administering the therapeutic agent at a concentration such that the therapeutic agent may not be found in the bloodstream of the subject. In some instances, the therapeutic agent cannot be detected in the cerebrospinal fluid of the subject. In some instances, the therapeutic agent cannot be detected in the urine of the subject. In some instances, the therapeutic agent is not administered at a concentration that it would be found in a solid organ other than the skin (e.g., liver, kidney, brain) in the subject. In some instances, the therapeutic agent is not administered at a concentration that it would be found on a cell surface of a cell, wherein the cell is part of a solid organ in the subject.

In some instances, methods comprise exposing only the epidermis to a composition disclosed herein, or a therapeutic agent thereof. In some instances, methods do not comprise exposing the dermis to a composition disclosed herein, or a therapeutic agent thereof. In some instances, compositions, or therapeutic agents thereof, disclosed herein do not move past the stratum granulosum when applied topically. In some instances, compositions, or therapeutic agents thereof, disclosed herein do not move past the basal cell layer of the epidermis when applied topically. In some instances, less than 1% of a therapeutic agent in the composition moves past the epidermis. In some instances, less than 5% of a therapeutic agent in the composition moves past the epidermis. In some instances, less than 10% of a therapeutic agent in the composition moves past the epidermis. In some instances, less than 15% of a therapeutic agent in the composition moves past the epidermis. In some instances, less than 20% of a therapeutic agent in the composition moves past the epidermis. In some instances, the therapeutic agent has a therapeutic effect on a skin disorder through activity on/in cells of the epidermis, but not cells in any other layer of the skin. In some instances, the therapeutic agent has a therapeutic effect on a skin disorder through activity on/in cells of the epidermis, but not cells beyond the skin in its entirety. In some instances, the cells are immune cells, (e.g., T-cells, macrophages or a combination thereof). In some instances, the therapeutic agent has a therapeutic effect on a skin disorder through activity on terminal nerve endings in the epidermis. In some instances, the therapeutic agent has a therapeutic effect on a skin disorder through activity on terminal nerve endings in the epidermis, but not any other layer of the skin. In general, transdermal delivery is avoided in order to keep plasma levels of the therapeutic agent minimal, negligible or zero.

In some instances methods comprise administering a pharmaceutical composition disclosed herein, such that the concentration of the therapeutic agent in the suitable carrier is low enough so as not to activate or potentiate a GABA-R on an immune cell of the subject, wherein the immune cell is not present in the skin of the subject. In some instances methods comprise administering a pharmaceutical composition disclosed herein, such that the concentration of the therapeutic agent in the suitable carrier is low enough so as not to activate or potentiate a GABA_{A} receptor on an immune cell of the subject, wherein the immune cell is not present in the skin of the subject. The immune cell may be an immune cell that is resident in the brain of the subject. The immune cell may be a circulating immune cell. The circulating immune cell may be an immune cell in the bloodstream. Non-limiting examples of immune cells include leukocytes, lymphocytes, B cells, T cells, and monocytes.

In some instances methods comprise administering a pharmaceutical composition disclosed herein, such that the concentration of the therapeutic agent in the suitable carrier is low enough so as not to activate or potentiate a GABA-R on a neuron of the subject, wherein the neuron is not present in the skin of the subject. In some instances methods comprise administering a pharmaceutical composition disclosed herein, such that the concentration of the therapeutic agent in the suitable carrier is low enough so as not to activate or potentiate a GABA_{A} receptor on a neuron of the subject, wherein the neuron is not present in the skin of the subject. The neuron may be a neuron in a muscle of the subject. The neuron may be a neuron in the brain of the subject.

In some instances, methods comprise administering a pharmaceutical composition disclosed herein, such that the concentration of the therapeutic agent in the suitable carrier is high enough to act as an agonist of a GABA_{A} receptor on a cell present in the skin of the subject. The cell present in the skin of the subject may be a neuron. The cell present in the skin of the subject may be an immune cell. The cell present in the skin of the subject may be is a T cell. In some instances, methods comprise administering a therapeutic agent disclosed herein, such that the concentration of the therapeutic agent in the suitable carrier is high enough to reduce proliferation of T cells in the skin. In some instances, methods comprise administering a therapeutic agent disclosed herein, such that the concentration of the therapeutic agent in the suitable carrier is high enough to reduce activation of T cells in the skin. In some instances, methods comprise administering a therapeutic agent disclosed herein, such that the concentration of the therapeutic agent in the suitable carrier is high enough to act as to reduce at least one of cytokine release and cytokine production by T cells of in the skin. In some instances, methods comprise administering a therapeutic agent disclosed herein, such that the concentration of the therapeutic agent in the suitable carrier is high enough to modulate activation of antigen presenting cells in the skin. In some instances, methods comprise administering a therapeutic agent disclosed herein, such that the concentration of the therapeutic agent in the suitable carrier is high enough to modulate macrophage migration in the skin.

Methods of treatment may comprise administering a pharmaceutical composition disclosed herein to an affected area at least once a day. Methods of treatment may comprise administering a pharmaceutical composition disclosed herein to an affected area at least twice a day. Methods of treatment may comprise administering a pharmaceutical composition disclosed herein to an affected area as needed. In some instances, methods comprise administering the pharmaceutical composition disclosed herein to an affected area at least once a week. In some instances, methods comprise administering the pharmaceutical composition disclosed herein to an affected area at least twice a week. In some instances, methods comprise administering the pharmaceutical composition disclosed herein to an affected area at least once a month. Methods of treatment disclosed herein may comprise administering the pharmaceutical composition to an affected area not more than once a day. Methods of treatment disclosed herein may comprise administering the pharmaceutical composition to an affected area not more than twice a day. Methods of treatment disclosed herein may comprise administering the pharmaceutical composition to an affected area not more than three times per day. Methods of treatment disclosed herein may comprise administering the pharmaceutical composition to an affected area once a day. Methods of treatment disclosed herein may comprise administering the pharmaceutical composition to an affected area twice a day. Methods of treatment disclosed herein may comprise administering the pharmaceutical composition to an affected area three times per day.

Methods of treatment may comprise administering a pharmaceutical composition disclosed herein to an affected area and allowing the pharmaceutical composition to remain on the affected area for a given duration, e.g., before washing the affected area or removing a bandage. In some instances, the duration is at least about 30 minutes. In some instances, the duration is at least about 1 hour. In some instances, the duration is at least about 2 hours. In some instances, the duration is at least about 4 hours. In some instances, the duration is about 30 minutes to at least about 1 hour. In some instances, the duration is about 1 hour to about 4 hours. In some instances, the duration is about 1 hour to about 12 hours.

Methods of treatment may comprise administering a pharmaceutical composition disclosed herein to an affected area by any number of various applications. In some instances, methods comprise rubbing the pharmaceutical composition on an affected area. In some instances, methods comprise spraying the pharmaceutical composition on an affected area. In some instances, methods comprise dropping the pharmaceutical composition on an affected area. In some instances, methods comprise wiping the pharmaceutical composition on an affected area. In some instances, methods comprise swabbing the pharmaceutical composition on to an affected area. In some instances, methods comprise dabbing the pharmaceutical composition on to an affected area. In some instances, methods comprise squeezing the pharmaceutical composition on to an affected area. In some instances, methods comprise administering a bandage to the subject, wherein the bandage comprises the pharmaceutical composition. In some instances, methods comprise administering the pharmaceutical composition to an affected area of the subject and further administering a bandage or cover to protect the treated area.

Propofol

Propofol may be a well-suited therapeutic agent for topical application with minimal or no transdermal delivery because of its size and lipophilicity. Propofol is an extremely lipophilic small molecule which helps it partition into the outer most layer of the skin called the stratum corneum, which comprises a lipid barrier. However, the viable epidermis is more aqueous and consequently its absorption becomes attenuated once it reaches the viable epidermis. This allows targeting of the epidermis while minimizing systemic transdermal absorption. This is an advantage of propofol over other GABA agonists that have less lipophilicity. In addition, propofol can depot into the stratum corneum, allowing the stratum corneum to serve as a drug reservoir for prolonged duration of effect. Prolonging the duration of action is important particularly when reducing the itch mediated by the terminal nerves in the skin. A sustained level of drug to continuously depolarize the nerve ending and limit nerve signaling would be desired.

Throughout the instant application, compositions of propofol and methods of administering propofol are described. It should be understood that derivatives and alternatives of propofol may be substituted for propofol in any instance, unless specified otherwise. Derivatives of propofol include, but are not limited to, fospropofol, HX0969w, PF0713, AZD-3043, and phaxan (alphaxalone). While some of these have been shown to be unsuitable for systemic administration (e.g., anesthetic purposes), they may be well-suited to topical application.

Propofol may be characterized by a high log P, “P” being “partition-coefficient.” In some instances, propofol has a log P greater than 3.5. In some instances, propofol has a log P greater than 4. Compounds with such high a log P tend to partition and stay in the epidermis which is more lipophilic. Hence propofol is idea for topical treatment, but not transdermal delivery. In contrast, drugs with intermediate log P (2-3) are best for systemic availability and transdermal delivery.

Propofol by itself delivered topically to subject is unique in that the subject would have limited systemic exposure and effects at formula concentration levels ranging from 1% to 20% w/w. Rationale for this range of concentrations is provided in Example 5. Plasma steady state levels of propofol of 1,000-2,000 ng/ml are used to maintain and anesthetized state. Wakening from propofol anesthesia occurs at plasma levels approximately less than 500 μg/ml. Minimal systemic side effects would be expected to occur at plasma levels 1000 times less than the anesthetic levels and 100 times less than the awakening level. Thus, desirable plasma levels of propofol in subjects being treated by methods disclosed herein and with composition disclosed herein are approximately less than 5 ng/ml. In some instances, plasma levels of propofol in subjects being treated by methods disclosed herein and with composition disclosed herein are not more than 10 ng/ml.

In some instances, methods of treatment comprise administering a pharmaceutical composition disclosed herein comprising a therapeutic agent, wherein the therapeutic agent is propofol. In some instances, methods comprise administering propofol to the skin of a subject in need thereof. In some instances, methods comprise administering propofol to an eye of a subject in need thereof. In some instances, the concentration of propofol in the suitable carrier is about 0.1% to about 50%. In some instances, the concentration of propofol in the suitable carrier is about 0.1% to about 5%, about 1% to about 5%, about 1% to about 10%, about 1% to about 20%, about 5% to about 25%, about 5% to about 40%, about 5% to about 50%, about 10% to about 25%, about 10% to about 40%, about 10% to about 50%, about 20% to about 30%, about 20% to about 40%, about 20% to about 50%, about 25% to about 35%, about 25% to about 45%, about 25% to about 50%, about 30% to about 40%, about 30% to about 50%, or about 40% to about 50%.

In some instances, methods of treatment comprise administering a pharmaceutical composition disclosed herein comprising a therapeutic agent, wherein the therapeutic agent is propofol. In some instances, the concentration of propofol in the suitable carrier is about 5%. In some instances, the concentration of propofol in the suitable carrier is about 10%. In some instances, the concentration of propofol in the suitable carrier is about 15%. In some instances, the concentration of propofol in the suitable carrier is about 20%. In some instances, the concentration of propofol in the suitable carrier is about 25%. In some instances, the concentration of propofol in the suitable carrier is about 30%. In some instances, the concentration of propofol in the suitable carrier is about 35%. In some instances, the concentration of propofol in the suitable carrier is about 40%. In some instances, the concentration of propofol in the suitable carrier is about 45%. In some instances, the concentration of propofol in the suitable carrier is about 50%.

Methods may comprise administering a pharmaceutical composition disclosed herein, wherein the pharmaceutical composition comprises propofol. According to description provided herein, systemic exposure to propofol should be minimal due to its high lipophilicity. Therefore, methods may comprise administering the pharmaceutical composition to an affected area 1-3 times daily. In some instances, methods comprise administering the pharmaceutical composition to an affected area 2-4 times daily. In some instances, methods comprise administering the pharmaceutical composition to an affected area 1-5 times daily. In some instances, methods comprise administering the pharmaceutical composition to an affected area until resolution of the condition, or a symptom thereof. If tachyphylaxis occurs, then methods may comprise administering for one week, followed by not administering for one week. Methods may comprise administering for 3-5 days, followed by not administering for 3-5 days. Methods may comprise administering for 2-3 days, followed by not administering for 2-3 days, as is done with topical glucocorticoids.

### Combination Therapies

Topical application of a GABA_{A}R modulator like propofol can cause itching, burning pain (nociception), and neurogenic inflammation (wheal and flare reaction). It has been shown that volatile anesthetics (e.g. isoflurane or sevoflurane), which also act on GABA_{A}R, when administered systemically, enhance ear swelling in an similar mouse ear inflammatory model that uses AITC (ref. Matta J A et. al., PNAS, 2008, 105(25):8784-8789. These symptoms may be mild, moderate or severe. Nociceptive effects may last approximately 5 to 10 minutes. Neurogenic inflammation may last approximately 90 minutes. These effects may be greatest at propofol concentrations of 10% w/w and above. These effects may be enhanced with the use of high concentrations of the penetration enhancers (e.g., DMSO), which causes a rapid delivery of propofol into the skin. Creams and ointment formulations may be helpful in limiting this effect. Surprisingly, despite activation of these inflammatory effects propofol reduces ear swelling and inflammation in an oxalobenzone induced mouse ear inflammatory model (see Example 2). This indicates the anti-inflammatory effects of propofol eventually overcome the acute effects of itching, burning pain, and neurogenic inflammation. However, it may be desirable to avoid, prevent or reduce these acute effects. Thus, methods of applying a combination of agents that avoid, prevent or reduce these effects are disclosed herein.

Disclosed herein are methods of delivering a GABA_{A}R modulator in combination with an additional therapeutic agent. The GABA_{A}R modulator may be an agonist of a GABA_{A}R beta subunit. The additional therapeutic agent may be a GABA-R modulator. The additional therapeutic agent may be a GABA_{A}R modulator. The additional therapeutic agent may be a GABA_{B}R modulator. The additional therapeutic agent may not be a GABA-R modulator. Exemplary GABA-R modulators are disclosed herein and throughout.

The aforementioned inflammatory and nociceptive effects are due to activation of transient receptor potential (TRP) channels, primarily TRPA1 and perhaps some TRPV1. Fortunately, stimulation of these receptors leads to down regulation and desensitization of these channels consistent with the reduced effects on repeated application. Desensitization caused by propofol has therapeutic benefits by desensitizing these channels. It is similar to the use of capsaicin, which activates the TRPV1 channel, and is used to treat pain and itch.

Methods disclosed herein may comprise delivering a TRP channel modulator to the subject in order to reduce initial or acute inflammatory or nociceptive effects due to delivery of the GABA_{A}R modulator. The TRP channel modulator may modulate the activity of the TRP channel. Methods may comprise delivering the TRP channel modulator before delivering the GABA_{A}R modulator to the subject. Methods may comprise delivering the TRP channel modulator and the GABA_{A}R modulator simultaneously to the subject. Methods may comprise delivering the TRP channel modulator after delivering the GABA_{A}R modulator to the subject.

Methods may comprise delivering a TRP channel modulator during initial treatment with a GABA_{A}R modulator. Methods may comprise ceasing delivery of the TRP channel modulator once TRP channels are desensitized to the GABA_{A}R modulator. TRP channels may be desensitized to the GABA_{A}R modulator after a first treatment with the GABA_{A}R modulator. TRP channels may be desensitized to the GABA_{A}R modulator after a first and second treatment with the GABA_{A}R modulator. TRP channels may be desensitized to the GABA_{A}R modulator after three treatments with the GABA_{A}R modulator. TRP channels may be desensitized to the GABA_{A}R modulator after five treatments with the GABA_{A}R modulator. TRP channels may be desensitized to the GABA_{A}R modulator after ten treatments with the GABA_{A}R modulator.

In some instances, the TRP channel modulator is a TRPA1 channel modulator. In some instances, the TRP channel modulator is a TRPV1 channel modulator. In some instances, the TRPV1 channel modulator is TRPV1-6. In some instances, the TRP channel modulator is a TRP channel antagonist. In some instances, the TRP channel modulator is a TRP channel agonist. In some instances, the TRP channel antagonist is a TRPA1 channel antagonist. In some instances, the TRPA1 channel antagonist is camphor. In some instances, the TRPA1 channel antagonist is a synthetic derivative of camphor. In some instances, the TRPA1 channel antagonist is a synthetic derivative of camphor, wherein the synthetic derivative is camphor substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino. In some instances, the TRPA1 channel antagonist is menthol. In some instances, the TRPA1 channel antagonist is a synthetic derivative of menthol. In some instances, the TRPA1 channel antagonist is a synthetic derivative of menthol, wherein the synthetic derivative is menthol substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino. In some instances, the TRP channel antagonist is a TRPV1 channel antagonist. In some instances, the TRP channel antagonist is a TRPM8 channel agonist. In some instances, the TRP channel antagonist is a TRPA1 channel antagonist and a TRPV1 channel antagonist. For example, menthol may act as an antagonist of both the TRPA1 channel and the TRPV1 channel. Topical application of camphor or menthol before or simultaneously with topical propofol may reduce nociception and neurogenic inflammation effects.

Additional modulators of TRPV1 that may be useful alone or in combination in methods and compositions disclosed herein include, but are not limited to, capsaicin and resiniferatoxin. Heat and acidity are also known to modulate TRPV1. Thus, compositions disclosed herein may comprise an agent with an acidity less than about pH 6, and methods disclosed herein may comprise a use thereof. In some instances, methods include applying heat to the affected area. TRPV1 is also modulated by vanilloids, lipoxygenase metabolites, prostaglandins, histamines, bradykinin, serotonin, and fatty acids, any of which may be used alone or in combination in compositions and methods disclosed herein to treat a skin condition.

Additional modulators of TRPA1 that may be useful in methods and compositions disclosed herein include, but are not limited to, isothiocyanates, cinnemaldehyde, iodoacetamide, acrolein and allicin. Isothiocyanates are found in horseradish and mustard. Allicin is found in garlic. Thus extracts and/or oils from these plants may be useful in compositions and methods disclosed herein. TRPA1 is also modulated by prostaglandins, nitrate fatty acids, arachidonic acid, diacylclycerol, and cannabinoids (e.g., cannabidiol, cannabichromene), any of which may be used alone or in combination in compositions and methods disclosed herein to treat a skin condition.

Modulators of TRPM8 include, but are not limited to menthol, icilin, and eucalyptol, all of which mimic a cooling sensation and may be desirable to a subject with a skin condition. Lysophospholipids may potentiate the effects of these ligands on TRPM8. Thus, methods disclosed herein may comprise applying to a subject in need thereof at least one agent selected from menthol, icilin, eucalyptol, a lysophospholipid, and a combination thereof.

In some instances, the TRP channel modulator is a GABA_{B}R agonist. The GABA_{B}R agonist may modulate the sensitivity of TRP channels, particularly TRPV1 and TRPA1 channels. Non-limiting examples of suitable GABA_{B}R agonists are baclofen, phenibut, isovaline, gamma-hydroxybutyrate, 3-aminopropylphosphinic acid, lesogaberan, 3-aminopropyl(methyl)phosphinic acid, CGP-44532, and a combination thereof. In some instances, the GABA_{B}R agonist is baclofen. In some instances, the GABA_{B}R agonist is a synthetic derivative of baclofen. In some instances, the GABA_{B}R agonist is a synthetic derivative of baclofen, wherein the synthetic derivative is baclofen substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino. In some instances, the GABA_{B}R agonist is phenibut. In some instances, the GABA_{B}R agonist is a synthetic derivative of phenibut. In some instances, the GABA_{B}R agonist is a synthetic derivative of phenibut, wherein the synthetic derivative is phenibut substituted by one or more groups independently selected from alkyl, alkenyl, alkynyl, cycloalkyl, aryl, heteroaryl, heterocycloalkyl, hydroxy, alkoxy, aryloxy, alkylthio, arylthio, alkylsulfoxide, arylsulfoxide, alkylsulfone, arylsulfone, sulfonamide, cyano, halogen, deuterium, acyl, carboxy, carboxyalkyl, nitro, haloalkyl, amido, amino, and alkylamino.

In some instances, the TRP channel modulator is a GABA_{B}R positive allosteric modulator. Non-limiting examples of suitable GABA_{B}R positive allosteric modulators are CGP-7930, BHFF ((R,S)-5,7-di-tert-butyl-3-hydroxy-3-trifluoromethyl-3H-benzofuran-2-one), fendiline, BHF-177, BSPP (2,6-di-tert-butyl-4-(3-hydroxy-2-spiropentylpropyl)-phenol), and GS-39783.

Methods may comprise delivering at least one TRP channel modulator and/or at least one GABA_{B}R agonist to the subject. Combination treatment with at least one TRP channel modulator and/or at least one GABA_{B}R agonist may improve tolerance to higher doses of the GABA_{A}R modulator and/or more frequent treatment of GABA_{A}R modulator (e.g., >2× per day application), relative to treatment with the GABA_{A}R modulator alone.

In some instances, methods comprise delivering a GABA_{A}R modulator, a TRP channel modulator, and a GABA_{B}R agonist to the subject. This combination treatment may improve tolerance to higher doses of the GABA_{A}R modulator and/or more frequent treatment of GABA_{A}R modulator (e.g., >2× per day application), relative to treatment with GABA_{A}R modulator alone or GABA_{A}R modulator with only one additional therapeutic reagent.

Pharmaceutical Compositions

Disclosed herein, in some aspects are pharmaceutical compositions that comprise a GABA-R modulator and a suitable carrier for topically applying the composition to an affected area of a subject. The GABA-R modulator may be a GABA-R agonist. The GABA-R modulator may be a direct GABA-R agonist. The GABA-R modulator may be a GABA_{A} receptor agonist. The GABA-R modulator may be a GABA_{B} receptor agonist. The affected area may be skin. The affected area may be an eye.

In some instances, the GABA-R modulator is a GABA_{A} receptor agonist. Non-limiting examples of GABA_{A} receptor agonists are barbiturates (at high local concentration), bamaluzole GABA, gabamide, GABOB, gaboxadol, ibotenic acid, isoguvacine, isonipecotic acid, muscimol, phenibut, picamilon, progabide, propofol (at high local concentration), quisqualamine, SL 75102, thiomuscimol, and zolpidem.

In some instances, the GABA-R modulator is a GABA_{A} receptor allosteric modulator. For instance, naturally occurring GABA binds and activates GABA_{A} receptor resulting in opening of the GABA_{A} receptor ion channel, thereby allowing ions into the neuron. This causes the neuron, on which the GABA_{A} receptor is present, to be less likely to fire. Thus, GABA typically reduces pain. itch and irritation. An allosteric regulator of GABA_{A} receptor increases the effects of GABA by making the channel open more frequently and or keeping the channel opening longer. Typically, the allosteric regulator has no effect on a GABA_{A} receptor if GABA or another GABA_{A} receptor agonist is not present. Non-limiting examples of GABA_{A} receptor allosteric modulators are alcohol, avermectins, barbiturates (at low doses), benzodiazepines, bromides, carbamates, chloralose, chlormezanone, clomethiazole, dihydroergolines, etazepine, etifoxine, imidazoles (e.g., etomidate), kavalactones, loreclezole, allopregnanolone, ganaxolone, zaleplon, zolpidem, zopiclone, eszopiclone, petrichloral, phenols (e.g, propofol at lower local concentration), piperidinediones, propanidid, pyrazolopyridines, quinazolinones, stripentol, sulfonylalkanes, valeric acid, and valerenic acids.

Propofol is also ideal because it has low oral bioavailability (<10%). Thus, a drug product with propofol provided for topical or ocular use would not lead to adverse events if ingested accidentally or on purpose. In contrast other GABA agonists and allosteric modulators have much higher oral bioavailability and may cause adverse events if ingested.

In some instances, the GABA-R modulator is a GABA_{B}R agonist. Non-limiting examples of GABA_{B}R agonists are 1,4-butanediol, baclofen, GABA, gabamide, GABOB, gamma-butyrolactone, gamma-hydroxybutyric acid, gamma-hydroxyvaleric acid, gamma-valerolactone, lesogaberan, phenibut, picamilon, progabide, SL-75102, and tolgabide.

In some instances, pharmaceutical compositions disclosed herein comprise a therapeutic agent, wherein the concentration of the therapeutic agent in the pharmaceutical composition is about 1% to about 25% w/w. In some instances, the concentration is at least one of about 1% to about 5%, about 5% to about 10%, about 10% to about 15%, about 15% to about 20%, and about 20% to about 25%.

In some instances, pharmaceutical compositions disclosed herein comprise propofol, wherein the concentration of propofol in the pharmaceutical composition is about 1% to about 25% w/w. The upper end of this range provides for a sufficiently high local concentration of propofol to activate both GABA_{A}R and GABA_{B}R in the epidermis. In some instances, the concentration is about 1% w/w to about 5% w/w in the composition. In some instances, the concentration is about 5% w/w to about 10% w/w in the composition. In some instances, the concentration is about 8% w/w to about 12% w/w in the composition. In some instances, the concentration is about 10% w/w to about 15% w/w in the composition. In some instances, the concentration is about 15% w/w to about 20% w/w in the composition. In some instances, the concentration is about 18% w/w to about 22% w/w in the composition. In some instances, the concentration is about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, or about 5% w/w in the composition. In some instances, the concentration is about 1% w/w in the composition. In some instances, the concentration is about 2% w/w in the composition. In some instances, the concentration is about 10% w/w in the composition. In some instances, the concentration is about 20% w/w in the composition.

In some instances, pharmaceutical compositions disclosed herein comprise a therapeutic agent, wherein the therapeutic agent is propofol. In addition to description provided elsewhere herein, propofol has properties unique to GABA-R modulators that make it particularly advantageous for methods and compositions disclosed herein. For instance, GABA alone is hydrophilic (as opposed to propofol which is lipophilic) and rapidly metabolized by GABA transaminase. GABA transaminase is abundant in the skin so GABA would not be suitable for topical application because it would not have a sufficient duration of activity. However, propofol is not metabolized by this transaminase. Gabapentin is not an effective GABA_{A}R or GABA_{B}R agonist. Thus, it would also not be suitable for methods and compositions disclosed herein. Most other GABA agonists do not have the lipophilicity property of propofol and are not as suitable for topical administrations and targeting the epidermis.

Combination Compositions

Pharmaceutical compositions disclosed herein may comprise delivering a GABA-R modulator and an additional agent. The GABA-R modulator may be a GABA_{A}R modulator. The additional agent may have therapeutic effects on the skin condition. The additional agent may have analgesic effects. The additional agent may have analgesic effects that aid in relieving discomfort caused by the GABA-R modulator. The additional agent may reduce initial or acute inflammatory or nociceptive effects due to delivery of a GABA_{A}R modulator. The additional agent may have minimal therapeutic effect on the skin condition. The additional agent may not have any therapeutic effect on the skin condition. The additional agent may be a GABA-R modulator disclosed herein. The additional agent may not be a GABA-R modulator disclosed herein. The additional agent may be a GABA_{A}R modulator disclosed herein. The additional agent may be a GABA_{B}R modulator disclosed herein. The additional agent may be a GABA_{B}R agonist disclosed herein. The additional agent may be a GABA_{B}R antagonist disclosed herein.

Pharmaceutical compositions disclosed herein may comprise an additional agent, wherein the additional agent is a transient receptor potential (TRP) channel modulator. In some instances, the TRP channel modulator is a TRPA1 channel modulator. In some instances, the TRP channel modulator is a TRPV1 channel modulator. In some instances, the TRPV1 channel modulator is TRPV1-6. In some instances, the TRP channel modulator is a TRP channel antagonist. In some instances, the TRP channel modulator is a TRP channel agonist. In some instances, the TRP channel antagonist is a TRPA1 channel antagonist. The TRPA1 channel antagonist may be camphor. The TRPA1 channel antagonist may be menthol. In some instances, the TRP channel antagonist is a TRPV1 channel antagonist. In some instances, the TRP channel antagonist is a TRPM8 channel agonist. In some instances, the TRP channel antagonist is a TRPA1 channel antagonist and a TRPV1 channel antagonist. For example, menthol may act as an antagonist of both the TRPA1 channel and the TRPV1 channel. Topical application of camphor or menthol before or simultaneously with topical propofol may reduce nociception and neurogenic inflammation effects.

Additional modulators of TRPV1 include, but are not limited to, capsaicin and resiniferatoxin. Acidity is known to modulate TRPV1. Thus, compositions disclosed herein may comprise an agent with an acidity less than about pH 6. TRPV1 is also modulated by vanilloids, lipoxygenase metabolites, prostaglandins, histamines, bradykinin, erotonin, and fatty acids, any of which may be used alone or in combination in compositions disclosed herein to treat a skin condition.

Additional modulators of TRPA1 that may be useful in compositions disclosed herein include, but are not limited to, isothiocyanates, cinnemaldehyde, iodoacetamide, acrolein and allicin. Isothiocyanates are found in horseradish and mustard. Allicin is found in garlic. Thus extracts and/or oils from these plants may be useful in compositions disclosed herein. TRPA1 is also modulated by prostaglandins, nitrate fatty acids, arachidonic acid, diacylclycerol, and cannabinoids (e.g., cannabidiol, cannabichromene), any of which may be used alone or in combination in compositions disclosed herein to treat a skin condition.

Modulators of TRPM8 include, but are not limited to menthol, icilin, and eucalyptol, all of which mimic a cooling sensation and may be desirable to a subject with a skin condition. Lysophospholipids may potentiate the effects of these ligands on TRPM8. Thus, pharmaceutical compositions disclosed herein may include menthol, icilin, eucalyptol, a lysophospholipid, or a combination thereof.

In some instances, pharmaceutical compositions disclosed herein comprise a GABA_{B}R agonist. The GABA_{B}R agonist may modulate the sensitivity of TRP channels, particularly TRPV1 and TRPA1 channels. Non-limiting examples of suitable GABA_{B}R agonists are baclofen, phenibut, isovaline, gamma-hydroxybutyrate, 3-aminopropylphosphinic acid, lesogaberan, 3-aminopropyl(methyl)phosphinic acid, CGP-44532, and a combination thereof.

In some instances, pharmaceutical compositions disclosed herein comprise a GABA_{B}R positive allosteric modulator. Non-limiting examples of suitable GABA_{B}R positive allosteric modulators are CGP-7930, BHFF ((R,S)-5,7-di-tert-butyl-3-hydroxy-3-trifluoromethyl-3H-benzofuran-2-one), fendiline, BHF-177, BSPP (2,6-di-tert-butyl-4-(3-hydroxy-2-spiropentylpropyl)-phenol), and GS-39783.

### Formulations

Pharmaceutical compositions disclosed herein may be presented in various formulations. In some instances, an appropriate base for a formulation disclosed herein is an ointment or cream. The ointment or cream may contain longer chain hydrocarbons, such as those found in petrolatum, paraffin waxes, beeswaxes, cetyl esters, mineral oil, triglycerides, lanolin, and steroids. The ointment or cream may comprise mineral oil. The ointment or cream may have a viscosity similar to petroleum jelly or honey at room temperature. The ointment or cream may prolong the duration of action after application by slowing the release of the propofol into the skin. Non-limiting examples of formulations include, Lipoderm®, Medihol™ gel base, VersaPro™ (lotion or cream), HRT Base™ (gel or cream), and Vanish-Pen™ Cream base. Another formulation may comprise Dermabase™. Dermabase™ is an oil-in-water formulation using mineral oil. Propofol is stable in Dermabase™ up to a concentration of 20% w/w.

As shown in Example 1, some formulations will not be suitable for certain concentrations of propofol. For example, compositions with concentrations of propofol greater than about 10% w/w may require a formulation with a higher alkane, e.g., mineral oil. Propofol is highly lipophilic and at higher concentrations requires another lipophilic substance like a higher alkane to be emulsified or stable in the composition. Otherwise the propofol may separate from the rest of the composition. For clarity, a higher alkane is characterized as having nine or more carbon atoms. A higher alkane may have up to about fifty carbon atoms. As shown in Example, 1, higher concentrations of propofol are stable in Dermabase™ cream. This is likely because Dermabase™ cream contains mineral oil. In contrast, propofol was not stable in Lipoderm® at 10% w/w. Lipoderm® is a mineral oil-free formulation. Mineral oil is also referred to as white oil, paraffin oil, and liquid petroleum. Alternatively or additionally to mineral oil and higher alkane containing substances, long chain fatty acids, or esters thereof, may be useful in emulsifying propofol when it is present at higher concentrations. Non-limiting examples of such fatty acids are oleic acid, linoleic acid, linolenic oil, palmitoleic oil, palmitic oil, stearic oil. These fatty acids are also found alone or in combination in naturally occurring oils, such as almond oil, safflower oil, sunflower oil, jojoba seed oil, orange peel oil, olive oil and coconut oil. Thus, composistions disclosed herein may comprise one of these naturally occurring oils.

Cosolvent penetration enhancers such as propylene glycol are often used by the pharmaceutical industry to enhance penetration of a therapeutic agent to deeper layers of the skin or for systemic absorption of lipophilic drugs. The primary, but not necessarily exclusive, purpose of the cosolvent penetration enhancer is to promote release of the drug from the base so that it partitions into the stratum corneum. However, compositions disclosed herein minimize penetration enhancers, including cosolvent penetration enhancers, in order to prevent systemic exposure of the therapeutic agent. In some instances, compositions do not comprise a penetration enhancer. A suitable amount of a cosolvent penetration enhancer may be about 1% to about 25%. In some instances, compositions disclosed herein comprise less than about 1% w/w penetration enhancer. In some instances, compositions disclosed herein comprise less than about 5% w/w penetration enhancer. In some instances, compositions disclosed herein comprise less than about 10% w/w penetration enhancer.

Often, topical formulations on the market comprise an agent that disrupts the stratum corneum to enhance penetration of a therapeutic agent to deeper layers of the skin or for systemic absorption of lipophilic drugs. Such disrupting agents include, but are not limited to, ethanol, methanol, DMSO, terpenes, and surfactants such as sodium dodecyl sulfate (SDS). Other surfactants that may be used in pharmaceutical compositions disclosed herein, either alone or in a combination are stearyl alcohol, sodium lauryl sulfate, cetyl alcohols, glycerol stearate, and cetyl esters. In particular, ethanol or other small terminal carbon alcohols (e.g. methanol) may be avoided as it can irritate the skin when combined with propofol possibly due to the formation of phenoxyl radicals. Formulations disclosed herein typically avoid, exclude or minimize such penetration agents. Formulations disclosed herein may have less then about 0.1%, less than about 0.5%, less than about 1%, less than about 5% or less than about 10% of such penetration agents.

In some instances, compositions disclosed herein rigid lipid nanoparticles such as rigid liposomes, solid lipid nanoparticles (SLNs), and nanostructured lipid carriers. These lipid carriers are either solid or rigid at both body and ambient temperature. These may aid in reducing systemic exposure, targeting the epidermis, prolonging duration of action, or a combination thereof. Exemplary lipids which are used in preparation of SLNs include fatty acids, steroids, waxes, monoglycerides, diglycerides and triglycerides. The size of the particles may range from about 50 nm to about 1000 nm. These nanoparticles partition into the stratum corneum and epidermis exceptionally well due to size and barrier effects. They can target the epidermis as their transcutaneous absorption declines when they encounter the hydrophilic viable epidermis. By way of non-limiting example propofol may easily incorporate into a lipophilic core of a solid lipid nanoparticle. Once absorbed to the epidermis the SLN releases the propofol over a prolonged period of time. Other lipid microemulsions or liposomes may be used, with variable residence times. Chitosan-based nanoparticles may also be used to encapsulate the drug and deliver it to the epidermis.

Pharmaceutical compositions disclosed herein are generally suitable for topical application. In some instances, pharmaceutical compositions disclosed herein are suitable for topical application to skin. In some instances, the composition is a formulation selected from a gel, ointment, lotion, emulsion, paste, cream, foam, mousse, liquid, spray, suspension, dispersion and aerosol. In some instances, the formulation comprises one or more excipients to provide a desired form and a desired viscosity, flow or other physical or chemical characteristic for effective application, coverage and adhesion to skin.

An emulsion may be described as a preparation of one liquid distributed in small globules throughout the body of a second liquid. In some embodiments, the dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A lotion may be described as a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A cream may be described as a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An ointment may be described as a semisolid preparation containing an ointment base and optionally one or more active agents of this disclosure. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A gel may be described as a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alkene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams may be described as an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Emollients may be described as externally applied agents that soften or soothe skin and are generally known in the art and listed in compendia, such as the “Handbook of Pharmaceutical Excipients”, 4.sup.th Ed., Pharmaceutical Press, 2003. In certain embodiments, the emollients are almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

Surfactants are surface-active agents that lower surface tension and thereby increase the emulsifying, foaming, dispersing, spreading and wetting properties of a product. In certain embodiments, suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

Emulsifiers are surface active substances which promote the suspension of one liquid in another and promote the formation of a stable mixture, or emulsion, of oil and water. In certain embodiments, the emulsifiers are metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate. In one embodiment, the emulsifier is glycerol. In one embodiment, the emulsifier is glycerin.

In some embodiments, compositions disclosed herein are formulated to be applied to a subject's scalp. In some embodiments, the composition is formulated to be used as a product selected from a shampoo, a conditioner, a mousse, a gel, and a spray. Such compositions would be useful for the treatment of seborrheic dermatitis. Treatment of seborrheic dermatitis with such compositions may result in the reduction of a symptom selected from dandruff and cradle cap. However, compositions disclosed herein may be used to treat seborrheic dermatitis at other areas of the body besides the scalp. Non-limiting examples of other areas include the chest, stomach, skin folds, arms, legs, groin area and under breasts.

In some embodiments, compositions disclosed herein comprise a buffer, wherein the buffer controls a pH of the composition. Preferably, the buffers buffer the composition from a pH of about 4 to a pH of about 7.5, from a pH of about 4 to a pH of about 7, and from a pH of about 5 to a pH of about 7.

In some embodiments, compositions disclosed herein are formulated to provide or maintain a desirable skin pH. In some embodiments, the desirable skin pH is between about 4.5 and about 6.5. In some embodiments, the desirable skin pH is between about 5 and about 6. In some embodiments, the desirable skin pH is about 5.5. In some embodiments, compositions disclosed herein are formulated with a skin pH modulating agent. Non-limiting examples of pH modulating agents include salicylic acid, glycolic acid, trichloroacetic acid, azeilic acid, lactic acid, aspartic acid, hydrochloride, stearic acid, glyceryl stearate, cetyl palmitate, urea phosphate, and tocopheryl acetate.

In some embodiments, compositions disclosed herein are formulated to provide more oxygen to the skin. In some embodiments, compositions disclosed herein are formulated to provide more oxygen exposure to the skin. In some embodiments, compositions disclosed herein are formulated to provide more oxygen diffusion into the skin. In some embodiments, compositions disclosed herein are formulated to provide more oxygen diffusion through the skin. In some embodiments, compositions disclosed herein are formulated with an agent that provides more oxygen to the skin. In some embodiments, compositions disclosed herein are used with an agent that provides more oxygen to the skin. In some embodiments, compositions disclosed herein are used before use of an agent that provides more oxygen to the skin. In some embodiments, compositions disclosed herein are used after use of an agent that provides more oxygen to the skin. A non-limiting example of an agent that provides oxygen to the skin is chlorophyll.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal. In one embodiment, a concentration of a preservative that is effective to prevent fungal growth is selected, without affecting the effectiveness of the composition for its intended purposed upon topical application.

Excipients in the formulation are selected based on the type of formulation intended. In certain embodiments, the excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

Compositions disclosed herein may be presented in a formulation that includes one or more excipients to improve any one or more of shelf-life, application, and therapeutic effect. In some embodiments, the excipient is necessary to improve any one or more of shelf-life, application, and therapeutic effect.

Pharmaceutical compositions disclosed herein are generally not intended for transdermal delivery, and thus, generally do not include skin penetration enhancers. Skin penetration enhancers are frequently used to promote transdermal delivery of drugs across the skin, in particular across the stratum corneum. Some penetration enhancers cause dermal irritation, dermal toxicity and dermal allergies. However, the more commonly used ones include urea, (carbonyldiamide), imidurea, N,N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10) oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

Compositions disclosed herein may be presented in a formulation that includes one or more excipients to reduce skin penetration of a therapeutic agent. Non-limiting examples of excipients that may reduce skin penetration of the therapeutic agent ointment excipients such as petrolatum, paraffin wax, steroids, glycerides, lanolin. Propofol will be more slowly released from these vehicle carriers and slow absorption. Rigid lipid nanoparticles may also be suitable for reducing skin penetration of the therapeutic agent. For reasons already described herein, ethanol and other small alcohols are typically avoided.

After formulation, compositions disclosed herein may be packaged in a manner suitable for delivery and use by an end user. In one embodiment, the composition is placed into an appropriate dispenser and shipped to the end user. Examples of a final container may include a pump bottle, squeeze bottle, jar, tube, capsule or vial.

In some embodiments, compositions disclosed herein can be added to an applicator before packaging. Non-limiting examples of applicators include a cotton pad, a polyester pad, a q-tip, a sponge, and a brush. In some embodiments, the applicator is placed in a package. Non-limiting examples of a package includes bags and foil or wax lined paper packets. The interior of the package may be sterile. In some embodiments, air in the package is removed with a vacuum before sealing. In some embodiments, the package is heat-sealed. In some embodiments, the package is sealed with adhesive.

Compositions disclosed herein may comprise additional active ingredients. Further disclosed are kits and systems that comprise a pharmaceutical composition disclosed herein and an additional active ingredient. Thus, the pharmaceutical composition and the additional active ingredient do not have to be packaged together or administered simultaneously. In some instances, the additional active ingredient and the pharmaceutical composition are packaged separately. In some instances, the additional active ingredient and the pharmaceutical composition are administered sequentially. Non-limiting examples of additional active ingredients are a small molecule, a fatty acid, an antibiotic, a probiotic, an antioxidant, a polypeptide, a vitamin or metabolite thereof, a steroid and a retinoid. In some embodiments, the additional active ingredient is an agent that has anti-inflammatory activity.

In some embodiments, the additional active ingredient comprises an antibiotic. Non-limiting examples of antibiotics include macrolide, tetracycline, β-lactam, aminoglycoside, cephalosporin, carbapenems, quinolone/fluoroquinolone, sulfonamides, salicylic acid, glycolic acid, azaleic acid, live phage therapy, synthetic phage contractile nanotubes, laser, dapsone, benzoyl peroxide, benzoyl peroxide/resveratrol combinations, and any combination thereof. In some embodiments, the antibiotic is selected from clindamycin, doxycycline, erythromycin, and tetracycline, wherein the antibiotic is formulated for topical administration.

In some embodiments, the additional active ingredient comprises a topical anti-acne medication such as benzoyl peroxide or salicylic acid. In some embodiments, the additional active ingredient comprises a topical anti-acne medication such as a retinoid. Non-limiting examples of topical retinoid compounds include retinoic acid, tretinoin, adapalene, and tazarotene. In certain embodiments, compositions disclosed herein comprise resveratrol or trans-resveratrol.

In some embodiments, the additional active ingredient comprises an omega-3 fatty acid. Non-limiting examples of omega-3 fatty acids include hexadecatrienoic acid (HTA), α-Linolenic acid (ALA), stearidonic acid (SDA), eicosatrienoic acid (ETE), eicosatetraenoic acid (ETA), eicosapentaenoic acid (EPA), heneicosapentaenoic acid (HPA), docosapentaenoic acid (DPA), clupanodonic acid, docosahexaenoic acid (DHA), tetracosapentaenoic acid, tetracosahexaenoic acid (nisinic acid), and phytosphingosine.

In some embodiments, the pharmaceutical composition is a formulation for administration to the eye. By way, of non-limiting example, the therapeutic agent of the pharmaceutical composition may be suspended in a saline solution or buffer that is suitable for dropping into the eye. In some embodiments, the pharmaceutical composition may be formulated as an eye drop, a gel, an oil, a lotion, an ointment, a suspension or an emulsion. For example, a gel or ointment may be applied under or inside an eyelid or in a corner of the eye.

For eye formulations comprising propofol, it is important to have a lipid carrier for the propofol. Thus, aqueous emulsions would be suitable for propofol. Emulsion droplets may be provided on a macro-, micro- or nano-scale. In contrast, aqueous gelling agents like hyaluronic acid would not be suitable for ocular formulations of propofol, as propofol would not be soluble in such gelling agents.

In some embodiments, the formulation for administration to the eye comprises a thickening agent, surfactant, wetting agent, base ingredient, carrier, excipient or salt that makes it suitable for administration to the eye. In some embodiments, the formulation for administration to the eye has a pH, salt or tonicity that makes it suitable for administration to the eye. These aspects of formulations for administration to the eye are described herein. In some embodiments, the pharmaceutical composition is an ophthalmic preparation. The pharmaceutical composition may comprise a thickening agent in order to prolong contact time of the pharmaceutical composition and the eye. In some embodiments, the thickening agent is selected from polyvinyl alcohol, polyethylene glycol, methyl cellulose, carboxy methyl cellulose, and combinations thereof. In some embodiments, the thickening agent is filtered and sterilized.

The pharmaceutical compositions disclosed herein may comprise a pharmaceutically acceptable carrier, pharmaceutically acceptable excipient or pharmaceutically acceptable salt for the eye. Non-limiting examples of pharmaceutically acceptable carriers, pharmaceutically acceptable excipients and pharmaceutically acceptable salts for they eye, include boric acid, calcium chloride, sodium perborate, phophonic acid, potassium chloride, magnesium chloride, sodium borate, sodium phosphate, and sodium chloride

The pharmaceutical compositions disclosed herein should be isotonic with lachrymal secretions. In some embodiments, the pharmaceutical composition has a tonicity from 0.5-2% NaCl. In some embodiments, the pharmaceutical composition comprises an isotonic vehicle. By way of non-limiting example, an isotonic vehicle may comprise boric acid or monobasic sodium phosphate.

In some embodiments, the pharmaceutical composition has a pH from about 3 to about 8. In some embodiments, the pharmaceutical composition has a pH from about 3 to about 7. In some embodiments, the pharmaceutical composition has a pH from about 4 to about 7. Pharmaceutical compositions outside this pH range may irritate the eye or form particulates in the eye when administered.

In some embodiments, the pharmaceutical compositions disclosed herein comprise a surfactant or wetting agent. Non-limiting examples of a surfactant employed in the pharmaceutical compositions disclosed herein are venzalkonium chloride, polysorbate 20, polysorbate 80, and dioctyl sodium sulpho succinate.

In some embodiments, the pharmaceutical compositions disclosed herein comprise a preservative that prevents microbial contamination after a container holding the pharmaceutical composition has been opened. In some embodiments, the preservative is selected from benzalkonium chloride, chlorobutanol, phenylmercuric acetate, chlorhexidine acetate, and phenylmercuric nitrate.

In some instances, pharmaceutical compositions disclosed herein comprise an additional active ingredient selected from an anti-inflammatory agent, an analgesic agent, an anti-itch agent, and a combination thereof.

Pharmaceutical compositions disclosed herein are generally intended for topical, localized treatment, without any significant systemic exposure to the subject treated therewith. Pharmaceutical composition disclosed herein may comprise a therapeutic agent at a concentration such that the therapeutic agent may not be found in a bodily fluid of the subject when the pharmaceutical composition is applied topically to the subject. The bodily fluid may be blood. The bodily fluid may be cerebrospinal fluid. The bodily fluid may be urine. In some instances, the pharmaceutical composition disclosed herein may comprise a therapeutic agent at a concentration such that it cannot be detected in a solid organ other than the skin (e.g., liver, kidney, brain) when the pharmaceutical composition is applied topically to the subject. In some instances, the pharmaceutical composition disclosed herein may comprise a therapeutic agent at a concentration such that it cannot be detected on a cell surface of a cell, wherein the cell is part of a solid organ, when the pharmaceutical composition is applied topically to the subject.

In some instances, pharmaceutical compositions comprise a therapeutic agent disclosed herein in a suitable carrier such that the concentration of the therapeutic agent in the suitable carrier is low enough so as not to activate or potentiate a GABA-R on an immune cell of the subject, wherein the immune cell is not present in the skin of the subject. In some instances, pharmaceutical compositions comprise a therapeutic agent disclosed herein in a suitable carrier such that the concentration of the therapeutic agent in the suitable carrier is low enough so as not to activate or potentiate a GABA_{A}R on an immune cell of the subject, wherein the immune cell is not present in the skin of the subject. The immune cell may be an immune cell that is resident in the brain of the subject. The immune cell may be a circulating immune cell. The circulating immune cell may be an immune cell in the bloodstream.

In some instances, pharmaceutical compositions comprise a therapeutic agent disclosed herein in a suitable carrier such that the concentration of the therapeutic agent in the suitable carrier is low enough so as not to activate or potentiate a GABA-R on a neuron of the subject, wherein the neuron is not present in the skin of the subject. In some instances, pharmaceutical compositions comprise a therapeutic agent disclosed herein in a suitable carrier such that the concentration of the therapeutic agent in the suitable carrier is low enough so as not to activate or potentiate a GABA_{A}R on a neuron of the subject, wherein the neuron is not present in the skin of the subject. The neuron may be a neuron in the brain of the subject. The neuron may be a neuron in a muscle of the subject.

In some instances, pharmaceutical compositions comprise a therapeutic agent disclosed herein in a suitable carrier such that the concentration of the therapeutic agent in the suitable carrier is high enough to act as an agonist of a GABA receptor on a cell present in the skin of the subject. The GABA receptor may be a GABA_{A}R. The GABA receptor may be a GABA_{B}R. The cell present in the skin of the subject may be a neuron. The cell present in the skin of the subject may be an immune cell. The cell present in the skin of the subject may be is a T cell. In some instances, pharmaceutical compositions comprise a therapeutic agent disclosed herein in a suitable carrier such that the concentration of the therapeutic agent in the suitable carrier is high enough to reduce proliferation of T cells in the skin. In some instances, pharmaceutical compositions comprise a therapeutic agent disclosed herein in a suitable carrier such that the concentration of the therapeutic agent in the suitable carrier is high enough to reduce activation of T cells in the skin. In some instances, pharmaceutical compositions comprise a therapeutic agent disclosed herein in a suitable carrier such that the concentration of the therapeutic agent in the suitable carrier is high enough to act as to reduce at least one of cytokine release and cytokine production by T cells of in the skin. In some instances, pharmaceutical compositions comprise a therapeutic agent disclosed herein in a suitable carrier such that the concentration of the therapeutic agent in the suitable carrier is high enough to modulate activation of antigen presenting cells in the skin. In some instances, pharmaceutical compositions comprise a therapeutic agent disclosed herein in a suitable carrier such that the concentration of the therapeutic agent in the suitable carrier is high enough to modulate macrophage migration in the skin.

In some instances, pharmaceutical compositions disclosed herein comprise a therapeutic agent in a suitable carrier, wherein the therapeutic agent is propofol. In some instances, the concentration of propofol in the suitable carrier is about 0.1% to about 50%. In some instances, the concentration of propofol in the suitable carrier is about 0.1% to about 5%, about 1% to about 5%, about 1% to about 10%, about 1% to about 20%, about 5% to about 25%, about 5% to about 40%, about 5% to about 50%, about 10% to about 25%, about 10% to about 40%, about 10% to about 50%, about 20% to about 30%, about 20% to about 40%, about 20% to about 50%, about 25% to about 35%, about 25% to about 45%, about 25% to about 50%, about 30% to about 40%, about 30% to about 50%, or about 40% to about 50%. In some instances, the concentration of propofol in the suitable carrier is about 5%. In some instances, the concentration of propofol in the suitable carrier is about 10%. In some instances, the concentration of propofol in the suitable carrier is about 15%. In some instances, the concentration of propofol in the suitable carrier is about 20%. In some instances, the concentration of propofol in the suitable carrier is about 25%. In some instances, the concentration of propofol in the suitable carrier is about 30%. In some instances, the concentration of propofol in the suitable carrier is about 35%. In some instances, the concentration of propofol in the suitable carrier is about 40%. In some instances, the concentration of propofol in the suitable carrier is about 45%. In some instances, the concentration of propofol in the suitable carrier is about 50%.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The examples and embodiments described herein are for illustrative purposes only and are not intended to limit the scope of the claims provided herein. Various modifications or changes suggested to persons skilled in the art are to be included within the spirit and purview of this application and scope of the appended claims.

### Example 1. Suitable Formulations of Propofol

Formulations of propofol were tested for stability. Stability was determined visually by separation of the emulsion and also analytically by changes in measured concentration of the cream and/or the formation of degradation products. Oil-in-water or water-in-oil formulations of propofol with a heavier oil or longer chain oil were stable. Heavier oils include petrolatum or mineral oil.

Propofol formulations were also stable when the amount of penetration enhancer in the formulation was limited to less than 10% w/w, particularly when the concentration of propofol is greater than 2%.

Concentrations of propofol>10% w/w were not stable in the following creams and lotions: Lipoderm®, Medihol™ gel base, HRT™ (gel or cream), VersaPro™ (lotion or cream), and Vanish-Pen™ Cream base.

Dermabase™ is an oil-in-water formulation using mineral oil. Propofol was stable in Dermabase™ up to 20% w/w propofol. Thus, this base, and its ingredients may be best suited for formulation with higher concentrations of propofol (>10% w/w). This stability at high concentrations may be due to the presence of mineral oil in Dermabase™. Ingredients in Dermabase™ include propylene glycol ˜10% w/w, and various surfactants, including stearyl alcohol, cetyl esters, cetyl alcohol, glycerol stearate, and sodium lauryl sulfate.

### Example 2. Topical Propofol Reduces Inflammation of Skin in a Mouse Model

Animals were sensitized to oxalbenzone on the abdomen on day 2 with 150 μl 3% oxalbenzone. Animals were challenged on day 5 with 1% oxalobenzone to both sides of one ear. This served as an in vivo pharmacological disease models for psoriasis and atopic dermatitis.

Several formulations of propofol were delivered at 100 μl to oxalobenzone treated ear at 1 hour and 6 hours post challenge. These formulations are as follows:
2% w/w propofol in DMSO
10% w/w propofol in DMSO
20% w/w propofol in DMSO
10% w/w propofol in Dermabase™ cream
vehicle (cream or DMSO)
dexamethasone in DMSO
10% w/w dexamethasone in Dermabase™ cream

DMSO was used as a control since it is easy to solubilize propofol in DMSO.

Ear thickness was measured with calipers at 24 hours and 48 hours and compared to contralateral non-challenged ear. Ear thickness served as an indication of swelling due to inflammation. The greater the ear thickness, the greater the inflammation.

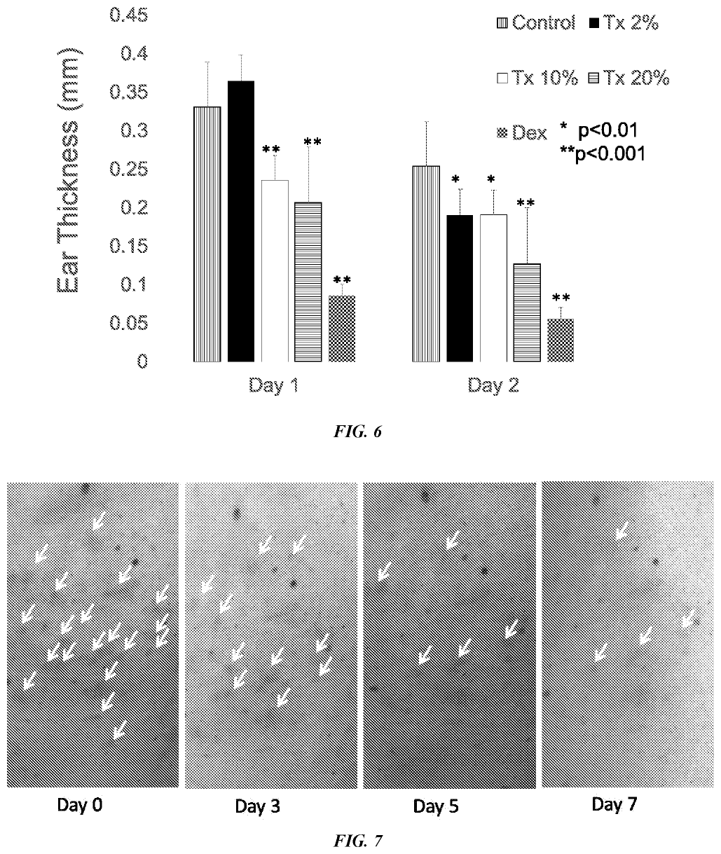

FIG. 6 shows control dexamethasone reduces inflammation (as measured by ear thickness), as expected. FIG. 6 also shows that both 10% and 20% propofol w/w in DMSO significantly reduces inflammation at 24 hours and 48 hours.

### Example 3. Topical Propofol Reduces Psoriatic Lesions in a Human Subject

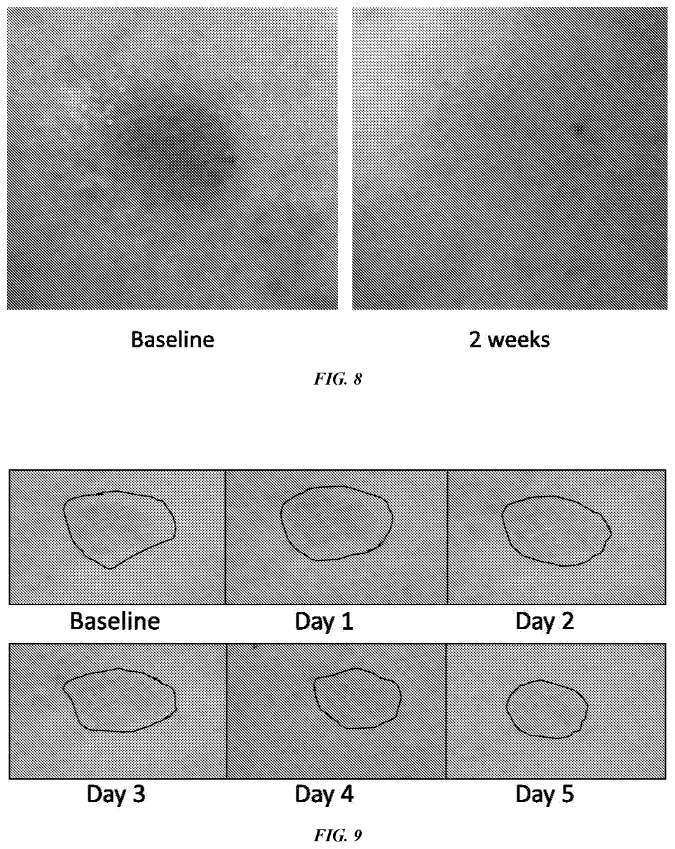

A human subject with psoriasis had topically applied with a 2% propofol solution twice daily. FIG. 7 shows that the number of psoriatic lesions were reduced after just three days of treatment and further reduced after a week of treatment.

The human subject had a gutate psoriatic lesion on his left lateral chest. This was treated with 10% propofol in cream base 2-3 times per day. The area of the psoriatic lesion steadily decreased over 5 days of treatment as shown in FIG. 8. Areas of the lesion are roughly outlined. When a second gutate psoriatic lesion on the left lateral chest was treated with 10% in cream base propofol once a day, the condition was completely resolved after two weeks of treatment. See FIG. 9.

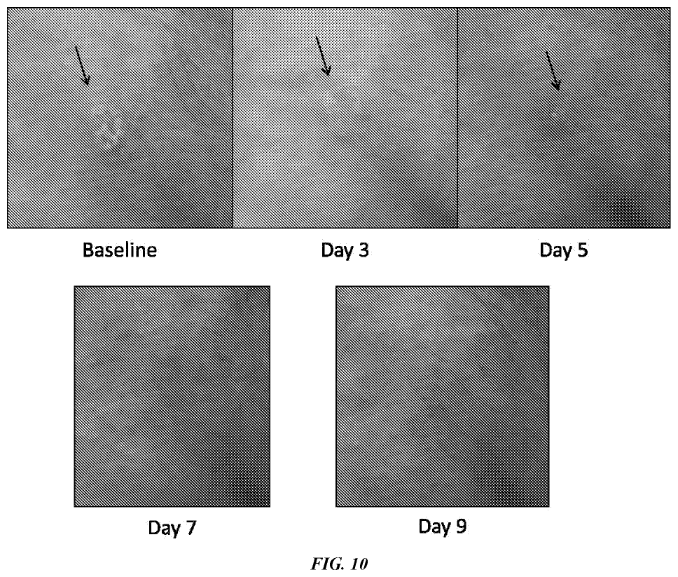

The human subject also had a plaque psoriatic lesion on their left forearm near their elbow. The plaque psoriatic lesion was raised and more lightly colored than the unaffected skin. This is shown in FIG. 10, see baseline. The plaque psoriatic lesion was treated with 10% propofol in cream twice daily. Over the course of treatment, the plaque psoriatic lesion diminished in height and area. After a week of treatment, the condition at this location was completely resolved.

Collectively, data collected showed local administration of propofol can produce anti-inflammatory effects locally; a sustained anti-nociceptive effect can be obtained, characterized by reduced sensation of itch and burning; repeated application of propofol resulted in less neurogenic inflammation; and topical application of propofol led to resolution of gutate and plaque psoriatic lesions. Based on these data, dosing at 1-2 times per day gives better results than dosing 3-4 times per day. This is likely due to reduced stimulation of neurogenic inflammation.

### Example 4. Topical Propofol Reduces Inflammation after an Initial Acute Reaction

Patches of skin on mice were individually treated with 10% propofol in DMSO, 10% propofol in a mineral oil containing cream, or control DMSO. 10% propofol in DMSO caused significant burning and inflammation about 10-15 minutes after treatment, as evidenced by redness with a wheal-flare effect. This effect was resolved after about 90 minutes. Mice showed a significant treatment effect despite this initial inflammatory response. 10% propofol in the mineral oil containing cream did not result in this effect.

### Example 5. Calculating Suitable Propofol Concentrations in Compositions for Topical Application

In rats, 500 mg of 100% propofol placed on the skin covering ˜3.5% body surface area in its native oily state can achieve plasma concentrations of 400 ng/ml. Formulations of a 20% w/w propofol in petrolatum (100 mg total dose of propofol) placed on ˜3.5% body surface area in rats can achieve plasma levels of 50-100 ng/ml (˜30 mg/1% body surface area). The high lipophilicity of propofol slightly reduces it release from the petrolatum and lowers the peak plasma concentration than what would be expected based on a linear extrapolation of total drug applied. Human equivalent plasma concentration levels based on this data are estimated to be 250 times lower than the rat plasma levels or ˜0.4-2 ng/ml well below the target of less than 5 ng/ml. Thus, as much as 20% w/w propofol in an ointment may be used to treat to 10%-20% body surface area in humans with-out causing significant systemic effects, even accounting for higher absorptions expected in inflamed skin. This would be suitable to treat mild to moderate dermatitis conditions.

As mentioned above a cosolvent enhancer may facilitate release of the propofol from the vehicle carrier. A 20% w/w formulation of propofol in propylene glycol, a cosolvent penetration enhancer, can increase the plasma concentration 2-3 fold to approximately 175-200 ng/ml. Addition of 30% w/w a surfactant penetration enhancer to the 20% propofol-propylene glycol formulation can increase the plasma concentration 3 to 5 fold to approximately 350 ng/ml. These would not be desirable plasma concentrations for methods disclosed herein. Thus, at least one of a surfactant penetration enhancer and cosolvent enhancer is minimized or avoided altogether.

### Example 6. Topical Propofol Treatment of Atopic Dermatitis

Atopic dermatitis is common in subjects with eczema. The Eczema Area and Severity Index (EAST) quantifies the severity of a subject's atopic dermatitis based on lesion severity and the percent of body surface area (BSA) affected. The EASI score ranges from 0.0 to 72.0 with higher scores corresponding to greater severity of atopic dermatitis.

The Physician's Global Assessment (PGA) assess the overall severity of atopic dermatitis. Scores range from 0 to 4, corresponding to severity (0=clear, 1=almost clear, 2=mild, 3=moderate, 4=severe.

Subjects with moderate to severe eczema, according to EASI and PGA, are treated with ointment containing propofol. Subjects are divided into groups, wherein each group receives a different propofol concentration. Group I receives ointment, wherein the concentration of propofol in the ointment is 50 mg/g. Group I receives ointment, wherein the concentration of propofol in the ointment is 100 mg/g. Group I receives ointment, wherein the concentration of propofol in the ointment is 200 mg/g. Propofol containing ointment is applied to the affected area of the skin at least once daily.

Subjects continue treatment for at least one week. Some subjects continue treatment for at least four weeks. Physicians examine the treated areas of the subjects and assess the effects of propofol using the EASI and PGA scores throughout treatment.

Blood samples are collected from the subjects to determine whether or not any significant amount of propofol has been absorbed. In some instances, blood samples are collected from the subjects to confirm that there is not a significant amount of propofol in the bloodstream. In some instances, blood samples are collected from the subjects to confirm that there is an absence of propofol in the bloodstream. In some instances, blood samples are collected from the subjects to confirm that there has been no systemic exposure to propofol.

### Example 7. Topical Propofol Treatment of Subjects with Ocular Inflammation

Subjects who have chosen to undergo cataract surgery are selected for a randomized, controlled study to evaluate the safety and efficacy of propofol treatment of the eyes. Subjects do not have a known hypersensitivity/contraindication to propofol. Nor do subjects have a history of glaucoma, intraocular pressure (IOP)>21 mmHg at the screening or randomization visits, or a history of being treated for glaucoma in either eye.

Subjects who undergo cataract surgery begin treatment with an ophthalmic suspension of propofol 24 hours after surgery. Subjects are dosed twice daily using an eye dropper containing the suspension. Treatment continues for one week. Subjects are asked to rate eye pain on a scale from 1 to 10. Attending physicians are asked to rate inflammation (redness and swelling) in the eye area on a scale from 1 to 10.

### Example 8. Topical Propofol Treatment of Subjects with Skin Ulcers

Subjects are diagnosed as having a chronic venous ulcer with localized non-systemic soft-tissue infection based on US FDA Guidance for Industry Chronic cutaneous ulcer and burn wounds. Generally, the ulcers in these patients are associated with diabetic foot syndrome. Ulcers are characterized by a combination of slough and necrotic tissue, exudate, smell, inflammation, presence of granulation tissue, and pain. Wound size is generally ≥5 cm2 and <200 cm2.

These subjects are selected for a randomized controlled trial that aims to provide evidence of the efficacy of propofol as monotherapy and combination therapy. The investigators designed a trial with two arms which are compared to each other. The treatment arms are as follows: (1) propofol alone and (2) propofol with antibiotic. Propofol alone or in combination with antibiotic are administered by spraying directly on to the site of the ulcer.

Patients receive treatment for 56 days, being dosed twice daily. After 28 and 56 days the wound surface are compared to the baseline. Infection parameters (c-reactive protein and leucocyte count) are measured weekly.

Primary outcome measure is reduction in wound area. Secondary outcome measures c-reactive protein levels, time to sterility of wound, relative wound volume reduction, and occurrence of drug resistant bacteria in the wound.

What is claimed is:

1) A method for treating a condition of the skin on a subject in need thereof comprising applying a therapeutic agent in a suitable carrier to an affected area of the skin, wherein the therapeutic agent is present in the suitable carrier at a concentration sufficient to act as an agonist of a gamma-amino-butyric-acid type A receptor (GABA_{A}R) beta subunit.

2) The method of claim 1, wherein the therapeutic agent acts as an agonist of GABA_{A}R beta subunit in the absence of gamma-amino-butyric-acid (GABA).

3) The method of claim 1 or 2, wherein the therapeutic agent acts as an agonist of a gamma-amino-butyric-acid type B receptor (GABA_{B}R).

4) The method of any preceding claim, wherein the therapeutic agent is lipophilic.

5) The method of any preceding claim, wherein the therapeutic agent is present in the suitable carrier at a concentration sufficient to behave as inhibitor of a fatty acid amide hydrolase.

6) The method of any preceding claim, wherein the therapeutic agent does not bind an alpha subunit of GABA_{A}R.

7) The method of any preceding claim, wherein the therapeutic agent does not bind a gamma subunit of GABA_{B}R.

8) The method of any preceding claim, wherein the therapeutic agent is non-steroidal.

9) The method of any preceding claim, wherein the therapeutic agent has a molecular weight of 500 Daltons or less.

10) The method of any preceding claim, wherein the therapeutic agent comprises propofol.

11) The method of any preceding claim, wherein the skin condition comprises inflammation.

12) The method of claim 11, wherein the skin condition is selected from dermatitis, psoriasis, eczema, hidradenitis suppurativa, and rosacea.

13) The method of claim 12, wherein dermatitis is selected from atopic dermatitis, contact dermatitis and seborrheic dermatitis.

14) The method of claim 13, wherein inflammation is associated with a Th2 mediated inflammatory disease.

15) The method of any preceding claim, wherein the condition of the skin comprises inflammation.

16) The method of any preceding claim, wherein the condition of the skin comprises skin itch.

17) The method of any preceding claim, wherein the condition of the skin comprises pain of the skin.

18) The method of claim 17, wherein the pain is associated with diabetic neuropathy or post herpetic neuralgia.

19) The method of any preceding claim, wherein the condition of the skin comprises a skin ulcer.

20) The method of claim 19, wherein the skin ulcer is a pressure ulcer.

21) The method of claim 19, wherein the skin ulcer is a vascular ulcer.

22) The method of any preceding claim, wherein the condition of the skin comprises a skin wound.

23) The method of any preceding claim, wherein the therapeutic agent does not activate or potentiate a GABA_{A} receptor on an immune cell of the subject other than an immune cell in the skin.

24) The method of any preceding claim, wherein the therapeutic agent does not penetrate the skin of the subject beyond the epidermis.

25) The method of any preceding claim, wherein the therapeutic agent reduces proliferation of T cells in the skin.

26) The method of any preceding claim, wherein the therapeutic agent reduces activation of T cells in the skin.

27) The method of any preceding claim, wherein the therapeutic agent reduces at least one of cytokine release and cytokine production by T cells of in the skin.

28) The method of any preceding claim, wherein the therapeutic agent modulates activation of antigen presenting cells in the skin.

29) The method of any preceding claim, wherein the therapeutic agent modulates macrophage migration in the skin.

30) The method of any preceding claim, wherein the concentration of the therapeutic agent in the pharmaceutical composition is about 1% w/w to about 25% w/w.

31) The method of any preceding claim, wherein the concentration of the therapeutic agent in the suitable carrier is about 5% w/w to about 20% w/w.

32) The method of any preceding claim, comprising applying the therapeutic agent more than once a day.

33) The method of any preceding claim, comprising applying the therapeutic agent more than twice a day.

34) A method for treating an ocular condition of a subject in need thereof comprising applying a therapeutic agent in a suitable carrier to an affected area of an eye of the subject, wherein the therapeutic agent is present in the suitable carrier at a concentration sufficient to act as an agonist of a GABA_{A}R beta subunit located on a cell in the affected eye area.

35) The method of claim 34, wherein the therapeutic agent acts as an agonist of GABA_{A}R beta subunit in the absence of gamma-amino-butyric-acid (GABA).

36) The method of claim 34 or 35, wherein the therapeutic agent acts as an agonist of a gamma-amino-butyric-acid type B receptor (GABA_{B}R).

37) The method of any one of claims 34-36, wherein the agonist is lipophilic.

38) The method of any one of claims 34-37, wherein the therapeutic agent is present in the suitable carrier at a concentration sufficient to behave as inhibitor of a fatty acid amide hydrolase.

39) The method of any one of claims 34-38, wherein the therapeutic agent does not bind an alpha subunit of GABA_{A}R.

40) The method of any one of claims 34-39, wherein the therapeutic agent does not bind an gamma subunit of GABA_{B}R.

41) The method of any one of claims 34-40, wherein the therapeutic agent is non-steroidal.

42) The method of any one of claims 34-41, wherein the therapeutic agent has a molecular weight of 500 Daltons or less.

43) The method of any one of claims 34-42, wherein the therapeutic agent is propofol.

44) The method of any one of claims 34-43, wherein the suitable carrier comprises a colloid.

45) The method of any one of claims 34-44, wherein the composition is in the form of an aqueous-oil emulsion.

46) The method of any one of claims 34-45, wherein the ocular condition is selected from ocular itch, ocular inflammation, a scratched cornea, conjunctivitis, an eye infection, and a combination thereof 47) The method of any one of claims 34-46, wherein applying comprises administering drops of the composition to the eye.

48) The method of any one of claims 34-47, wherein the suitable carrier is an ointment and applying comprises dabbing the composition along an edge or corner of an eyelid.

49) The method of any one of the preceding claims, wherein a plasma concentration of the therapeutic agent in the subject does not exceed 5 ng/ml.

50) A composition comprising a therapeutic agent in a suitable carrier for topical application, wherein the therapeutic agent is present in the suitable carrier at a concentration sufficient to act as an agonist of a GABA_{A}R beta subunit.

51) The composition of claim 50, wherein the therapeutic agent acts as an agonist of GABA_{A}R beta subunit in the absence of gamma-amino-butyric-acid (GABA).

52) The composition of claim 50 or 51, wherein the therapeutic agent acts as an agonist of a gamma-amino-butyric-acid type B receptor (GABA_{B}R).

53) The composition of any one of claims 50-52, wherein the therapeutic agent is lipophilic.

54) The composition of any one of claims 50-53, wherein the therapeutic agent is present in the suitable carrier at a concentration sufficient to behave as inhibitor of a fatty acid amide hydrolase.

55) The composition of any one of claims 50-54, wherein the therapeutic agent does not bind an alpha subunit of GABA_{A}R.

56) The composition of any one of claims 50-55, wherein the therapeutic agent does not bind a gamma subunit of GABA_{B}R.

57) The composition of any one of claims 50-56, wherein the therapeutic agent is non-steroidal.

58) The composition of any one of claims 50-57, wherein the therapeutic agent has a molecular weight of 500 Daltons or less.

59) The composition of any one of claims 50-58, wherein the therapeutic agent comprises propofol.

60) The composition of any one of claims 50-59, wherein the therapeutic agent does not activate or potentiate a GABA_{A} receptor on an immune cell of the subject other than an immune cell in the skin.

61) The composition of any one of claims 50-60, wherein the therapeutic agent does not penetrate the skin of the subject beyond the epidermis.

62) The composition of any one of claims 50-61, wherein the therapeutic agent reduces proliferation of T cells in the skin.

63) The composition of any one of claims 50-62, wherein the therapeutic agent reduces activation of T cells in the skin.

64) The composition of any one of claims 50-63, wherein the therapeutic agent reduces at least one of cytokine release and cytokine production by T cells of in the skin.

65) The composition of any one of claims 50-64, wherein the therapeutic agent modulates activation of antigen presenting cells in the skin.

66) The composition of any one of claims 50-65, wherein the therapeutic agent modulates macrophage migration in the skin.

67) The composition of any one of claims 50-66, wherein the concentration of the therapeutic agent in the pharmaceutical composition is about 1% w/w to about 25% w/w.

68) The composition of any one of claims 50-67, wherein the concentration of the therapeutic agent in the suitable carrier is about 5% w/w to about 20% w/w.

69) The composition any one of claims 50-68, wherein the suitable carrier is selected from at least one of a cream, an emulsion, an ointment, a microparticle, a nanoparticle, a suspension, a gel, a lotion, and a patch.

70) The composition any one of claims 50-69, wherein the composition comprises an additional active ingredient selected from an anti-inflammatory agent, an analgesic agent, an anti-itch agent, and a combination thereof.

71) The composition of any one of claims 50-70, wherein the composition does not comprise a skin penetration enhancer.

72) The composition of any one of claims 50-71, wherein the composition does not comprise at least one of ethanol, methanol, DMSO, terpene, and SDS.

73) The composition of any one of claims 50-72, wherein the composition contains mineral oil, a triglyceride, lanolin, and a steroid.

74) The composition of claim 73, wherein the composition contains mineral oil.

75) The composition of any one of claims 50-74, wherein the suitable carrier comprises at least one of a lipid nanoparticles and a liposome.

76) The composition of claim 75, wherein the suitable carrier maintains the composition as a solid at body temperature.

77) The composition of any one of claims 50-76, wherein the composition is formulated for administration to the eye.

78) The composition of claim 77, wherein the suitable carrier comprises a colloid.

79) The composition of claim 77, wherein the composition is in the form of an aqueous-oil emulsion.

80) The composition of any one of claims 50-79, wherein the therapeutic agent has a log partitioning coefficient greater than 4.

81) The composition of any one of claims 50-80, comprising a GABA_{B}R modulator.

82) The composition of any one of claims 50-81, comprising a TRP channel modulator.

83) A method for treating an inflammatory condition of the skin of a subject, the method comprising applying propofol in a suitable carrier to an affected area of the skin.

84) The method of claim 83, wherein the inflammatory condition comprises cytokine mediated inflammation, Th2 mediated inflammation, or a combination thereof.

85) The method of claim 83, wherein the inflammatory condition is selected from dermatitis psoriasis, eczema, hidradenitis suppurativa, rosacea, herpetic neuralgia, and a combination thereof.

86) The method of claim 85, wherein dermatitis comprises atopic dermatitis.

87) The method of claim 85, wherein dermatitis comprises contact dermatitis.

88) The method of claim 85, wherein dermatitis comprises seborrheic dermatitis.

89) The method of claim 83, wherein the suitable carrier is selected from a cream and an ointment.

90) The method of claim 83, wherein the suitable carrier comprises less than 10% w/w of a penetration enhancer.

91) The method of claim 83, wherein the concentration of propofol in the carrier is 0.1%-20% w/w.

92) The method of claim 83, comprising applying propofol in the suitable carrier 1 to 3 times per day.

93) A method for treating an inflammatory condition of the skin of a subject, the method comprising applying propofol and a GABA_{B}R modulator to an affected area of the skin.

94) The method of claim 93, wherein the GABA_{B}R modulator enhances, increases or promotes activity of GABA_{B}R.

95) The method of claim 93, wherein the GABA_{B}R modulator is a GABA_{B}R agonist.

96) The method of claim 93, wherein the propofol and GABA_{B}R modulator are applied as a single composition.

97) The method of claim 96, wherein the concentration of propofol in the single composition is 0.1%-20% w/w.

98) The method of claim 96, wherein the concentration of GABA_{B}R modulator in the single composition is 0.1%-10% w/w.

99) The method of claim 93, wherein the propofol is delivered in a first composition and the GABA_{B}R modulator is applied in a second composition.

100) The method of claim 99, wherein the first composition has a different pharmaceutical carrier than the second composition.

101) The method of claim 99, wherein at least one of the first composition and the second composition contains less than 10% w/w of a penetration enhancer.

102) The method of claim 99, wherein the concentration of propofol in the first composition is 0.1%-20% w/w.

103) The method of claim 99, wherein the concentration of GABA_{B}R modulator in the second composition is 0.1%-10% w/w.

104) The method of claim 93, wherein the inflammatory condition comprises Th2 mediated inflammation.

105) The method of claim 93, wherein the inflammatory condition is selected from dermatitis psoriasis, eczema, hidradenitis suppurativa, rosacea, herpetic neuralgia, and a combination thereof 106) The method of claim 105, wherein dermatitis comprises atopic dermatitis.

107) The method of claim 105, wherein dermatitis comprises contact dermatitis.

108) The method of claim 105, wherein dermatitis comprises seborrheic dermatitis.

109) The method of claim 93, wherein at least one of the propofol and the GABA_{B}R modulator is formulated as a cream or an ointment.

110) The method of claim 93, wherein the GABA_{B}R modulator is baclofen.

111) The method of claim 93, wherein the GABA_{B}R modulator is phenibut.

112) The method of claim 93, wherein applying does not occur more than 3 times per day.

113) A method for treating an inflammatory condition of the skin of a subject, the method comprising applying a GABA_{A}R modulator and a GABA_{B}R modulator to an affected area of the skin.

114) The method of claim 113, wherein the GABA_{B}R modulator enhances, increases or promotes activity of GABA_{B}R.

115) The method of claim 113, wherein the GABA_{B}R modulator is a GABA_{B}R agonist.

116) The method of claim 113, wherein the GABA_{A}R modulator and GABA_{B}R modulator are applied as a single composition.

117) The method of claim 116, wherein the concentration of GABA_{A}R modulator in the single composition is 0.1%-20% w/w.

118) The method of claim 116, wherein the concentration of GABA_{B}R modulator in the single composition is 0.1%-10% w/w.

119) The method of claim 113, wherein the GABA_{A}R modulator is delivered in a first composition and the GABA_{B}R modulator is applied in a second composition.

120) The method of claim 119, wherein the first composition has a different pharmaceutical carrier than the second composition.

121) The method of claim 119, wherein at least one of the first composition and the second composition contains less than 10% w/w of a penetration enhancer.

122) The method of claim 119, wherein the concentration of GABA_{A}R modulator in first composition is 0.1%-20% w/w.

123) The method of claim 119, wherein the concentration of GABA_{B}R modulator in second composition is 0.1%-10% w/w.

124) method of claim 113, wherein the inflammatory condition comprises Th2 mediated inflammation.

125) The method of claim 113, wherein the inflammatory condition is selected from dermatitis psoriasis, eczema, hidradenitis suppurativa, rosacea, herpetic neuralgia, and a combination thereof.

126) The method of claim 125, wherein dermatitis comprises atopic dermatitis.

127) The method of claim 125, wherein dermatitis comprises contact dermatitis.

128) The method of claim 125, wherein dermatitis comprises seborrheic dermatitis.

129) The method of claim 113, wherein at least one of the GABA_{A}R and the GABA_{B}R modulator is formulated as a cream or an ointment.

130) The method of claim 113, wherein the GABA_{B}R modulator is baclofen.

131) The method of claim 113, wherein the GABA_{B}R modulator is phenibut.

132) The method of claim 113, wherein applying does not occur more than 3 times per day.

133) The method of any one of claims 113-132, wherein the GABA_{A}R modulator is a GABA_{A}R agonist.

134) The method of any one of claims 113-132, wherein the GABA_{A}R modulator is a propofol.

135) The method of any one of claims 113-132, wherein the GABA_{A}R modulator is a barbiturate.

136) The method of any one of claims 113-132, wherein the GABA_{A}R modulator is a zolpidem.

137) A method for treating an inflammatory condition of the skin of a subject, the method comprising applying a GABA_{A}R modulator and a TRP channel modulator to an affected area of the skin.

138) The method of claim 137, wherein the TRP channel modulator and GABA_{A}R modulator are applied as a single composition.

139) The method of claim 138, wherein the concentration of GABA_{A}R modulator in the single composition is 0.1%-20% w/w.

140) The method of claim 138, wherein the concentration of TRP channel modulator in the single composition is 0.1%-10% w/w.

141) The method of claim 137, wherein the GABA_{A}R modulator is delivered in a first composition and the TRP channel modulator is applied in a second composition.

142) The method of claim 141, wherein the first composition has a different pharmaceutical carrier than the second composition.

143) The method of claim 141, wherein at least one of the first composition and the second composition contains less than 10% w/w of a penetration enhancer.

144) The method of claim 141, wherein the concentration of GABA_{A}R modulator in the first composition is 0.1%-20% w/w.

145) The method of claim 141, wherein the concentration of TRP channel modulator in the second composition is 0.1%-10% w/w.

146) The method of claim 137, wherein the inflammatory condition comprises Th2 mediated inflammation.

147) The method of claim 137, wherein the inflammatory condition is selected from dermatitis psoriasis, eczema, hidradenitis suppurativa, rosacea, herpetic neuralgia, and a combination thereof.

148) The method of claim 147, wherein dermatitis comprises atopic dermatitis.

149) The method of claim 147, wherein dermatitis comprises contact dermatitis.

150) The method of claim 147, wherein dermatitis comprises seborrheic dermatitis.

151) The method of claim 135, wherein at least one of the GABA_{A}R modulator and the TRP channel modulator is formulated as a cream or an ointment.

152) The method of any one of claims 135-151, wherein the TRP channel modulator is camphor.

153) The method of any one of claims 135-151, wherein the TRP channel modulator is menthol.

154) The method of claim 137, wherein applying occurs more than 2 times per day.

155) The method of any one of claims 137-154, wherein the GABA_{A}R modulator is a GABA_{A}R agonist.

156) The method of any one of claims 137-155, wherein the GABA_{A}R modulator is a propofol.

157) The method of any one of claims 137-155, wherein the GABA_{A}R modulator is a barbiturate.

158) The method of any one of claims 137-155, wherein the GABA_{A}R modulator is a zolpidem.

159) The method of any one of claims 137-158, wherein the TRP channel modulator is a TRP channel antagonist.

160) The method of any one of claims 137-159, wherein the TRP channel modulator modulates the activity of a TRP channel selected from TRPA1, TRPV1 and TRPM8.

161) The method of any one of claims 137-160, further comprising applying a GABA_{B}R modulator to the affected area of the skin.

162) The method of claim 161, wherein the GABA_{B}R modulator, GABA_{A}R modulator, and TRP channel modulator are applied as a single pharmaceutical composition.

163) The method of claim 161, wherein the GABA_{B}R modulator is applied before at least one of the GABA_{A}R modulator and the TRP channel modulator.

164) The method of claim 161, wherein the GABA_{B}R modulator is applied after at least one of the GABA_{A}R modulator and the TRP channel modulator.

165) A method for treating skin itch of a subject comprising applying a GABA_{A}R modulator and a GABA_{B}R modulator to affected areas of the skin.

166) The method of claim 165, wherein the GABA_{A}R modulator is propofol.

167) The method of claim 165, wherein the GABA_{B}R modulator and GABA_{A}R modulator are applied as a single composition.

168) The method of claim 167, wherein the concentration of GABA_{A}R modulator in the single composition is 0.1%-20% w/w.

169) The method of claim 167, wherein the concentration of GABA_{B}R modulator in the single composition is 0.1%-10% w/w.

170) The method of claim 165, wherein the GABA_{A}R modulator is delivered in a first composition and the GABA_{B}R modulator is applied in a second composition.

171) The method of claim 170, wherein the concentration of GABA_{A}R modulator in the first composition is 0.1%-20% w/w.

172) The method of claim 170, wherein the concentration of GABA_{B}R modulator in the second composition is 0.1%-10% w/w.

173) The method of claim 170, wherein the first composition has a different pharmaceutical carrier than the second composition.

174) The method of claim 165, wherein at least one of the GABA_{A}R modulator and GABA_{B}R modulator is applied in the form selected from a cream, emulsion, ointment, nanoparticle, lotion, patch, and a combination thereof.
